SEQUENCE LISTING (24 sequences)

SEQ ID NO: 1 - DNA, 18 bp, Artificial Sequence
  Synthetic TALEN
gctatctcgcagatacgg

SEQ ID NO: 2 - DNA, 15 bp, Artificial Sequence
  Synthetic TALEN
cactggacttcgaga

SEQ ID NO: 3 - DNA, 90 bp, Artificial Sequence
  Synthetic HDR Template
agctgctctgctatctcgcagatacggcccagaaaggccaagatctcacagtccagtgagccggtcactgtccccgaggtcccacactcc

SEQ ID NO: 4 - DNA, 90 bp, Artificial Sequence
  Synthetic HDR Template
agctgctctgctatctcgcagatacggcccagaaaggccaagatcttcaagtccagtgagccggtcactgtccccgaggtcccacactcc

SEQ ID NO: 5 - DNA, 90 bp, Artificial Sequence
  Synthetic HDR Template
agctgctctgctatctcgcagatacggcccagaaaggccaagatcttcatctccagtgagccggtcactgtccccgaggtcccacactcc

SEQ ID NO: 6 - DNA, 15 bp, Artificial Sequence
  Synthetic TALEN
cagtcatgcaatttt

SEQ ID NO: 7 - DNA, 16 bp, Artificial Sequence
  Synthetic TALEN
ccaattcccaagtaat

SEQ ID NO: 8 - DNA, 18 bp, Artificial Sequence
  Synthetic TALEN
gtcatatccataaaaaac

SEQ ID NO: 9 - DNA, 17 bp, Artificial Sequence
  Synthetic TALEN
gtcctaacattttatat

SEQ ID NO: 10 - DNA, 90 bp, Artificial Sequence
  Synthetic Oligo HDR Template
ccttggtatgtgatcagatcagtcatgcaattttcacttcatggatccgacacactatataaaatgttaggacatcagctcataaacaga

SEQ ID NO: 11 - DNA, 20 bp, Artificial Sequence
  Synthetic Primer Sequence
ctgaccatcgacgcttctga

SEQ ID NO: 12 - DNA, 20 bp, Artificial Sequence
  Synthetic primer sequence
aactcaacaacggcacctga

SEQ ID NO: 13 - DNA, 20 bp, Artificial Sequence
  synthetic primer sequence
cagatgcgcaccaaaaagct

SEQ ID NO: 14 - DNA, 20 bp, Artificial Sequence
  synthetic primer sequence
cagcccttcctaatgccctc

SEQ ID NO: 15 - DNA, 19 bp, Artificial Sequence
  Sythteic Nucleotide Sequence
tgaaggcaagaagacagac

SEQ ID NO: 16 - DNA, 19 bp, Artificial Sequence
  Synthetic nucleotide sequence
tggtcaaatactcttctat

SEQ ID NO: 17 - DNA, 63 bp, Sus scrofa
ctgctatctcgcagatacggcccagaaaggccacgatctcgaagtccagtgagccggtcatgt

SEQ ID NO: 18 - DNA, 64 bp, Artificial Sequence
  Synthetically alterted nucleotide
ctgctatctcgcagatacggcccagaaaggccaagatcttcatctccagtgagccggtcactgt

SEQ ID NO: 19 - DNA, 50 bp, Artificial Sequence
  Synthetic TALEN
atcagtcatgcaattttcacttcatgaaaagaattacttgggaattggat

SEQ ID NO: 20 - DNA, 55 bp, Artificial Sequence
  Synthetic TALEN
atgtcatatccataaaaaacaaatgtcagacacactatataaaatgttaggacat

SEQ ID NO: 21 - DNA, 55 bp, Artificial Sequence
  Sythetic TALEN
ttgaaggcaagaagacagacaaaaagtacctgatgatagaagagtatttgaccaa

SEQ ID NO: 22 - DNA, 70 bp, Sus scrofa
ggacaactttgaaggcaagaagacagacaaaaagtacctgatgatagaagagtatttgaccaaagagcta

SEQ ID NO: 23 - DNA, 70 bp, Artificial Sequence
  Synthetic oligo sequence
agacaactttgaaggcaagaagactgacaaaaagtacctgatgatcaaagagtatttgaccaaagagctg

SEQ ID NO: 24 - DNA, 70 bp, Artificial Sequence
  Synthetic oligo sequence
agacaactttgaaggcaagaacavtgacaaaaagtacctgatgatcgaagagtatttgaccaaagagctg

(19) United States
(12) Patent Application Publication
(10) Pub. No.: US 2018/0084767 A1
(43) Pub. Date: Mar. 29, 2018

(54) ANIMAL MODELS FOR CARDIOMYOPATHY

(71) Applicant: Recombinetics, Inc., Saint Paul, MN (US)

(72) Inventors: Daniel F. Carlson, Woodbury, MN (US); Scott C. Fahrenkrug, Minneapolis, MN (US); Dennis Webster, Saint Paul, MN (US)

(21) Appl. No.: 15/711,920

(22) Filed: Sep. 21, 2017

Related U.S. Application Data
(60) Provisional application No. 62/397,539, filed on Sep. 21, 2016.

(51) Int. Cl. A01K 67/027

(52) U.S. Cl. CPC A01K 67/0275; A01K 2227/108; A01K 2217/075; A01K 2267/0375

(57) ABSTRACT

Genomically modified livestock animals having a modification in one or more genes implicated in heart failure are provided. The animals provide models for various pathologies in heart failure including dilated cardiomyopathy and hypertrophic cardiomyopathy and can be used for investigation of new treatment methods including interventional devices, biologics and pharmaceuticals. The models can also be induced to develop metabolic syndrome (MetS) and are therefore amenable to further investigation of the confounding effects of MetS on the progress of heart failure.

### CROSS REFERENCE TO RELATED APPLICATIONS

This patent application claims priority to U.S. Provisional Patent Application No. 62/397,539, filed Sep. 21, 2016 and which is hereby incorporated by reference in its entirety.

### FIELD OF THE INVENTION

The invention is directed to genetically modified animal models useful in investigating cardiomyopathy, heart failure and therapies thereof.

### BACKGROUND OF THE INVENTION

Heart failure or heart disease are general terms denoting the inability of the heart to maintain an optimum cardiac output. In many instances, this deficiency of the heart is due to coronary artery disease, hypertension, diabetes and valvular heart disease. However, in many cases the deficiency is due to a pathology of the heart muscle itself. These various diseases are generally termed “cardiomyopathy”. In many cases, these pathologies result from Mendelian genetic disorders. Pathogenic heart failure and cardiomyopathy have traditionally been identified as a group of diseases including dilated cardiomyopathy (DCM), hypertrophic cardiomyopathy (HCM), restrictive cardiomyopathy (RCM), arrhythmogenic cardiomyopathy (AVC) and unclassified cardiomyopathy. In these pathologies, HCM and DCM make up, by far, the majority of CM cases.

The ultimate etiology of CM often cannot be determined. However, numerous factors can cause CM, including: genetic, congenital heart defects, infections, drug and alcohol abuse, cancer medications, exposure to toxins, coronary artery disease, high blood pressure, diabetes and complications of late-stage pregnancy. DCM and HCM make up, by far, the large majority of CM cases. Dilated cardiomyopathy (DCM) is a disease in which the heart ventricles become dilated and thinner and cannot pump blood efficiently. DCM is a progressive and debilitating disease that inevitably leads to a prototypical clinical presentation of HF, with impaired delivery of life-sustaining nutrients and oxygen to the body [10, 11]. Dilated cardiomyopathy is the most common form of non-ischemic cardiomyopathy. It occurs more frequently in men than in women, and is most common between the ages of 20 and 60 years.^{[2]} About one in three cases of congestive heart failure (CHF) is due to dilated cardiomyopathy.^{[1]} Dilated cardiomyopathy also occurs in children. The deterioration of cardiac function in DCM results in HF that can be refractory to medical therapy, leading to significant morbidity and need for mechanical devices or cardiac transplantation to prevent death [12, 13]. Recognition of DCM as a familial disorder, in up to 50% of cases, has been the impetus for human genetics investigations to uncover the molecular basis of DCM and develop suitable HF model systems [14, 15]. DCM has proved to be genetically heterogeneous, but myocardial degeneration and development of HF is the common final pathway and requires more effective clinical management strategies. HCM occurs when the cells of the heart muscle enlarge causing the walls of the ventricles to thicken while the ventricle size does not change thus decreasing the ejection volume of blood and causing obstruction of the coronary vessels. HCM is frequently asymptomatic until acute infarct occurs and is one of the most prevalent causes of death in young athletes. HCM is generally thought to be a monogenic disease caused by a mutation in one of 13 or more sarcomere genes. However, because the cellular pathogenesis of these diseases are not sufficiently understood to explain variable age-dependent penetrance, the ability to provide novel diagnostic, prognostic, or therapeutic tools is limited. Therefore, a large animal model system that recapitulates human CM pathogenesis would enable mechanistic investigation and provide a physiological system to test new therapeutic approaches for HF.

### SUMMARY OF THE INVENTION

The present invention provides animal models of HF useful in study the pathology and treatment of the disease.

Disclosed herein are genomically modified non-human animals comprising one or more mutations in genes linked to HF. These include but are not limited to alleles of: ANKRD1, BAG3, CAMK2D, CRYAB, CSRP3, DES, DMD, EYA4, GATAD1, ILK, LAMA4, LDB3, LMNA, MYBPC3, MYH6, MYH7, MYPN, PDLIM3, PLN, PSEN1/2, RBM20, RYR2, SCN5A, SGCD, TAZ/G4.5, TCAP, TMPO, TNNC1, TNNI3, TNNT2, TPM1, TIN and/or VCL.

In various exemplary embodiments, the mutation is in an RS rich region of the gene. In some exemplary embodiments, the modification is made with gene editing technology. In various embodiments, the gene editing technology includes TALENs, CRISPR/CAS9, ZFN, meganucleases. In some exemplary embodiments, the mutation in the allele is the only modification to the genome of the animal. In other embodiments, two or more specifically edited genes are the only modification to the genome of the animal. In these embodiments, the two or more genetic modifications are the result of a single multiplex gene editing event using multiple gene editing enzymes targeting different, specific loci. See, for example U.S. Pub App. 2016/0029604 hereby incorporated by reference for all purposes in its entirety. In other exemplary embodiments the two or more genetic modifications are the result of serial gene editing events with the two or more genetic modifications being in an animal being the result of serial breeding of F1 or their progeny combining separate gene editing events. In exemplary embodiments the animal is a livestock animal. In these embodiments the livestock animal includes a bovine, ovine or porcine. In various embodiments, the porcine animal is a minipig. In some exemplary embodiments the minipig is an Ossabaw minipig. In various exemplary embodiments, the modification is heterozygous. In some exemplary embodiments, the modification is homozygous. In exemplary embodiments, the modification is one or more alleles of ANKRD1, BAG3, CAMK2D, CRYAB, CSRP3, DES, DMD, EYA4, GATAD1, ILK, LAMA4, LDB3, LMNA, MYBPC3, MYH6, MYH7, MYPN, PDLIM3, PLN, PSEN1/2, RBM20, RYR2, SCN5A, SGCD, TAZ/G4.5, TCAP, TMPO, TNNC1, TNNI3, TNNT2, TPM1, TTN and/or VCL. In particular exemplary embodiments, the mutation includes: R636H, R636S or S635A relative to numbering on the human gene or introduced into TTN or BAG3.

In still other exemplary embodiments, the invention provides a method of making a non-human, animal-model for heart failure comprising modifying an animal genome to create a mutation in one or more of a ANKRD1, BAG3, CAMK2D, CRYAB, CSRP3, DES, DMD, EYA4, GATAD1, ILK, LAMA4, LDB3, LMNA, MYBPC3, MYH6, MYH7, MYPN, PDLIM3, PLN, PSEN1/2, RBM20, RYR2, SCN5A, SGCD, TAZ/G4.5, TCAP, TMPO, TNNC1, TNNI3, TNNT2, TPM1, TTN and/or VCL.

In various exemplary embodiments according to the invention the mutation is within a hotspot in the gene. In some exemplary embodiments the mutation is in the RS rich region of a gene. In these embodiments, the mutation is site specific with no other mutations in the genome except those in the one or more target genes. In exemplary embodiments, the modification is made with gene editing technology. In various embodiments, the gene editing technology includes TALENs, CRISPR/CAS9, ZFN and meganucleases. In some embodiments, the mutation in the allele is the only modification to the genome of the animal. In other exemplary embodiments, the animal has modification of more than one allele or gene or combination of alleles or genes. In these various embodiments, the animal may be heterozygous for the modification, homozygous for the modification, or compound heterozygous for the modification. In various exemplary embodiments, the invention provides a method for creating a suite of modified animals providing various genotypes for the investigation of cardiomyopathy comprising wild type animals, homozygously modified animals, compound heterozygote animals and heterozygote animals. In various embodiments the animal is a livestock animal. In these exemplary embodiments the livestock animal includes a goat, ovine or porcine. In various embodiments, the porcine animal is a minipig. In some embodiments the minipig is an Ossabaw minipig.

In still other exemplary embodiments an animal model of cardiomyopathy is provided according to the invention. In these exemplary embodiments, the animal model has a targeted modification of one or more genes implicated in cardiomyopathy. In various exemplary embodiments the model comprises a modification of one or more genes comprising ANKRD1, BAG3, CAMK2D, CRYAB, CSRP3, DES, DMD, EYA4, GATAD1, ILK, LAMA4, LDB3, LMNA, MYBPC3, MYH6, MYH7, MYPN, PDLIM3, PLN, PSEN1/2, RBM20, RYR2, SCN5A, SGCD, TAZ/G4.5, TCAP, TMPO, TNNC1, TNNI3, TNNT2, TPM1, TTN and/or VCL. In some embodiments the modification comprises homozygous, heterozygous and/or compound heterozygous modifications. In these and other embodiments the genetic modification is made using gene editing technology. In various embodiments, the gene editing technology includes TALENs, CRISPR/CAS9, ZFN and meganucleases. In some embodiments, the mutation in the allele is the only modification to the genome of the animal. In various embodiments according to the invention the animal model is used in clinical testing of drugs, biologics or devices to treat heart failure.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be apparent from the description, as set forth hereinafter.

### BRIEF DESCRIPTION OF THE DRAWINGS

FIG. 1: Using patient data and gene-editing to produce better swine models of human disease.

FIGS. 2A-2E: Hetero- and homozygous editing of R636S in swine. FIG. 2A) The porcine WT sequence for RBM20 displayed with TALENs (white arrows) designed to cleave near the target codon to convert R636 to serine. FIG. 2B) The same sequence after editing with TALENs and the HDR template. FIG. 2C) RFLP analysis on a population of cells treated with the TALENs and HDR template. FIG. 2D) RFLP analysis of individual clones derived from the transfected cells. The WT product is 392 bp, and the RFLP allele has cleavage products of 228 and 164 bp.

FIG. 2E) Sequence confirmation of hetero- and homozygous introgression of the R636S allele into pig fibroblasts. The clone number refers to the RFLP analysis in FIG. 2D.

FIGS. 3A-3C.: Founder genotyping reveals a KO allele and significant genotype effect on mortality. FIG. 3A) The schematic shows the expected amplicon from the primers (black arrows) and the size of the BglII cleavage products for the R636S allele. Below is a schematic representation of a large deletion that removes the splice acceptor for exon 9. FIG. 3B) PCR amplicons flanking the target site +/− restriction digest with BglII. Genotype notations are as follows; Wt wild-type; HTZ=heterozygous; CMPD=compound heterozygous; and HMZ=homozygous. FIG. 3C) Plot showing high rate of stillborn and mortality in the first week of life for homozygous animals and comparably little mortality in the heterozygous group. HMZ (n=21) HTZ (n=8) CMPD Het (n=9).

FIG. 4: Kaplan-Meier curve for RBM20 heterozygous, homozygous for the R636S mutation, and compound heterozygotes (R636S/−) with one KO allele and one R636S mutant allele. Wild-type swine have essentially 100% survival in the first 12 weeks of life without traumatic injury during the neonatal period. However, there is a strong dose dependent genotype/phenotype correlation with RBM20 mutations. Homozygous animals (solid black) have a ˜25% survival at 12-weeks with the majority of mortality occurring with sudden neonatal death. Heterozygous animals (large/small dash line) have ˜80% survival at 12-weeks. Survival of compound heterozygotes is intermediate compared to heterozygous and homozygous animals.

FIGS. 5A-5C: Cardiac histopathology of RBM20-R636S pigs. Tissue sections were stained with hematoxylin and eosin (H&E) (FIG. 5A) or Masson-trichrome stain (FIG. 5B). Bar=50 urn. Notice increased fibrosis in the RBM20-homozygous (HMZ) animals compared to wild type (WT) animals with extensive fibrosis at the endocardium (Endo) and septum (white (FIG. 5B). The photomicrograph in FIG. 5A (H&E staining), showing that the endocardium is thickened and composed of elastic fibers in the RBM20-HMZ animals. Endocardial fibrous area was quantified by cellSens Dimension software (FIG. 5C).

FIG. 6: Elevated levels of circulating cTnI and BNP in RBM20 mutants. Mean and standard error of serum cTnI and plasma BNP levels from 12 RBM20 mutant and 15 wild type pigs evaluated using the Pig Cardiac Tn-I, Ultra-Sensitive ELISA kit (Life Diagnostics) and non-competitive immunoradiometric assay (IRMA) methods for BNP. ** Means are significantly different (p<0.001).

FIGS. 7A-7H: Cardiac MRI imaging from 8-week-old piglets. Three-dimensional volumetric imaging analysis using MRI FIG. 7A) wild-type long-axis, FIGS. 7B-7C) RBM20 homozygous mutants long-axis, FIGS. 7D-7E) short-axis, and FIG. 7F) volumetric calculations.

FIGS. 7G-7H) RBM20 HMZ mutations (white bar) demonstrate a significant decrease in cardiac function in a double blinded study at 8-weeks of age compared to wild-type animals (black bar). Additionally, HTZ animals were statistically similar to wild-type at this age other than a measurable increase in heart rate (data not shown). *Student T-test with p<0.05 (n=4 per group) FIGS. 8A-8F: Elevated levels of circulating BNP and ANP in RBM20 homozygous mutants. FIGS. 8A-8B) Mean and standard error of serum BNP and ANP levels from 4 RBM20 mutant (edited) and 4 wild type pigs (non-edited) evaluated using non-competitive immunoradiometric assay (IRMA). FIGS. 8C-8D) Gross pathological samples at 8 weeks of age from wild-type and RBM20 homozygous mutant animal that suddenly died. FIGS. 8E-8F) Corresponding Tri-chrome stain demonstrating significant fibrosis in the RBM20 homozygous mutant animal. ** Means are significantly different (p<0.01).

FIG. 9: Germline stem cell transplantation (GST) to rescue failure-to-thrive phenotypes of R636S homozygotes. It is expected that the majority of R636S homozygotes will not be in sufficient health to serve as breeders, but will reach the age of 8-12 weeks when they are ideal donors for GST. Stem cells isolated from the R636S boars will be transplanted into age-matched DAZL-KO boars. DAZL-KO boars do not have any germ cells at 12 weeks, and absent spermatogenesis at 9 months of age-therefore will not transmit their own genetics. However, this environment provides an open niche for the transplanted germ cells to engraft, mature and produce sperm. Thus, only R636S sperm will be produced allowing model propagation from a healthy boar.

FIG. 10: Differential splicing of cardiac transcripts in RBM20 mutant swine. Homozygous null animals (HMZ) and compound heterozygous (CMPD) demonstrate expected isoform changes in CAMK2D with the both L and S isoforms. Furthermore, TTN demonstrates the classical changes with a longer isoform. This demonstrates the expected molecular changes.

FIG. 11: Hemodynamics on 24 week animals. This investigation utilizes the 24-week old animals in (above) to collect invasive measurements immediately prior to tissue collection for histology and RNA.

FIGS. 12A-12C: Event Recorders to document arrhythmia burden on each genotype. FIG. 12A) 5 kg piglets recovering from surgery. FIG. 12B) Medtronic clinical system. FIG. 12C) Calibrated swine readouts to document present events.

FIG. 13: Hemodynamics at 8 weeks. This investigation utilizes C-section delivered piglets to allow immediate application of ILR activity monitors will also be used that use implantable telemetry as needed. This cohort will also provide invasive hemodynamics for all three genotypes at 8 weeks.

FIGS. 14A-14C: Excision of Proximal Tandem Ig domains 3-11 of the porcine Titin (TTN) gene. FIG. 14A) TALEN pairs were designed to target the 5′ intron and 3′ intron of Proximal Tandem Ig domains 3 and 11, respectively, of ssTTN. FIG. 14B) Transfected TALEN mRNA targeting either the 5′ intron (5.1) or 3′ intron (3.1) showed an editing efficiency of 44.9% and 60.0% respectively. FIG. 14C) Cells co-transfected with both 5′ and 3′ TALEN mRNA plus an ssODN repair template were subjected to PCR analysis for deletion of the Ig domain. The resulting amplicon was the expected size (457 bp) following successful removal of Ig domains 3-11.

FIGS. 15A-15B: Introduction of E455K mutation into the porcine BAGS gene using TALENs. FIG. 15A) TALENs (ssBAG3 4.1) were designed to target ssBAG3 and single stranded oligonucleotides (ssODNs) were designed to introduce the E455K mutation (ha E455K oligo) as well as humanize (hsWT oligo) the region surrounding the desired mutation via Homology Directed Repair (HDR). The SNP responsible for E455K is indicated by an arrow, and additional humanizing base changes are indicated with arrow heads. FIG. 15B) When transfected, the TALENs showed and editing efficiency of 21.3%.

FIG. 16: Kaplan-Meier curve for compiled RBM20 WT and homozygotes for the R636S mutation. Some decrease in mortality, compared to FIG. 4, may result from more intensive animal management practices.

FIGS. 17A-17C: Three EKG strips from newborn HMZ piglets demonstrating various symptoms of heart disease. FIG. 17A. V-tachycardia; FIG. 17B. bradycardia (B-cardia) and FIG. 17C. complete block.

FIGS. 18A-18D: FIG. 18A) Cardiac MRI imaging from 8-week-old piglets. RBM20 HMZ mutations (dotted lines) demonstrate a significant decrease in cardiac function, in a double blinded study at 8-weeks of age compared to wild-type animals. FIG. 18B) Elevated levels of circulating BNP in RBM20 homozygous mutants. FIG. 18C and FIG. 18D) Three-dimensional volumetric imaging analysis using MRI to measure left ventricle end diastolic volume (LVEDS) and left ventricle end systolic volume (LVESV). ** Means are significantly different (p<0.001).

### DETAILED DESCRIPTION OF THE EXEMPLARY EMBODIMENTS

Heart failure or heart disease is a general term denoting the inability of the heart to maintain an optimum cardiac output. In many instances, this deficiency of the heart is due to coronary artery disease, hypertension, diabetes and valvular heart disease. However, in many cases the deficiency is due to a pathology of the heart muscle itself. These various diseases are termed “cardiomyopathy” (CM). In many cases, these pathologies result from Mendelian genetic disorders. Pathogenic heart failure and cardiomyopathy have traditionally been identified as a group of diseases including dilated cardiomyopathy (DCM), hypertrophic cardiomyopathy (HCM), restrictive cardiomyopathy (RCM), arrhythmogenic cardiomyopathy (AVC) and unclassified cardiomyopathy. In these pathologies, HCM and DCM make up, by far, the majority of CM cases.

DCM is a condition in which the heart becomes enlarged and cannot pump blood efficiently. When the heart chambers dilate, the heart muscle does not contract properly. Over time, the disease results in a vicious circle with the fatigue of the heart leading to increased dilation which results in a further decrease in the ability of the heart to contract. The disease often starts in the left ventricle and then spreads to the right ventricle and atria as the disease progresses. The decreased heart function can affect the lungs, liver, and other body systems. HCM is denoted by portions of the myocardium being enlarged without any obvious cause and can lead to sudden death. HCM occurs if heart muscle cells enlarge and cause the walls of the ventricles (usually the left ventricle) to thicken. Despite this thickening, the ventricle size often remains normal. However, the thickening may block blood flow out of the ventricle. If this happens, the condition is called obstructive hypertrophic cardiomyopathy. HCM is frequently asymptomatic until sudden cardiac arrest. RCM is characterized by the ventricles becoming stiff and rigid. This happens because abnormal tissue, such as scar tissue, replaces the normal heart muscle. As a result, the ventricles can't relax normally and fill with blood, and the atria become enlarged. Over time, blood flow in the heart is reduced. This can lead to problems such as heart failure or arrhythmias. RCM affects mostly older adults. AVC is a rare type of cardiomyopathy. AVC occurs if the muscle tissue in the right ventricle dies and is replaced with scar tissue. This process disrupts the heart's electrical signals and causes arrhythmias. Symptoms include palpitations and fainting after physical activity.

Cardiomyopathy is a major cause of heart failure (HF) and a significant source of mortality and morbidity for children and adults. There is no cure for CM. Advanced myocardial degeneration at the time of symptomatic presentation limits effectiveness of current pharmacologic therapies; consequently CM is the most common indication for heart transplantation. In addition, HCM is commonly asymptomatic until sudden cardiac arrest makes its occurrence known. Despite numerous genetic models in rodents, and induced models in large animals, the progress towards developing effective pharmaceutical treatments and interventive medicine has been slow for two primary reasons; 1) rodent and induced models do not accurately mimic disease progress and response to therapy in humans and 2) genetic heterogeneity of the disease results in a limited number of attractive molecular targets. Recently, mutations in various genes have been implicated as causative to HF. These include mutations in both structural and functional genes. These genes include, but are not limited to the genes provided in Table 1.

TABLE 1*

| Gene | Protein | Pig Ensembl ID |
|---|---|---|
| ABCC9 | SUR2A | ENSSSCG00000000571 |
| ACTC1 | Cardiac actin | ENSSSCG00000004803 |
| ACTN2 | α-actinin-2 | ENSSSCG00000010144 |
| ANKRD1 | Ankyrin repeat domain- | ENSSSCG00000010461 |
| | containing protein 1 | |
| BAG3 | BCL2-associated athanogene 3 | ENSSSCG00000010686 |
| CAMK2D | Calcium/Calmodulin Dependent | ENSSSCG00000009123 |
| | Protein Kinase II Delta | |
| CRYAB | Alpha B crystalin | ENSSSCG00000015025 |
| CSRP3 | Muscle LIM protein | ENSSSCG00000013354 |
| DES | desmin | ENSSSCG00000020785 |
| DMD | Dystrophin | ENSSSCG00000028148 |
| EYA4 | Eyes-absent 4 | ENSSSCG00000023510 |
| GATAD1 | GATA zinc finger domain | ENSSSCG00000025577 |
| | containing 1 | |
| ILK | Integrin-linked kinase | ENSSSCG00000023272 |
| LAMA4 | Laminin a-4 | ENSSSCG00000004425 |
| LDB3 | Cypher/ZASP | ENSSSCG00000010359 |
| LMNA | Lamin A/C | ENSSSCG00000006496 |
| MYBPC3 | Myosin-binding protein C | ENSSSCG00000013236 |
| MYH6 | α-myosin heavy chain | ENSSSCG00000030999 |
| MYH7 | β-myosin heavy chain | ENSSSCG00000002029 |
| MYPN | Myopalladin | ENSSSCG00000029311 |
| PDLIM3 | PDZ LIM domain protein 3 | ENSSSCG00000015796 |
| PLN | Phospholamban | ENSSSCG00000004248 |
| PSEN1/2 | Presenilin 1/2 | ENSSSCG00000002340/ |
| | | ENSSSCG00000010860 |
| RBM20 | RNA binding protein 20 | ENSSSCG00000010626 |
| RYR2 | ryanodine receptor 2 | ENSSSCG00000010142 |
| SCN5A | Sodium channel | ENSSSCG00000011259 |
| SGCD | δ-sarcoglycan | NA |
| TAZ/G4.5 | Tafazzin | NA |
| TCAP | Titin-cap or telethonin | ENSSSCG00000017500 |
| TMPO | Thymopoietin | ENSSSCG00000000887 |
| TNNC1 | Cardiac troponin C | ENSSSCG00000011441 |
| TNNI3 | Cardiac troponin I | ENSSSCG00000024505 |
| TNNT2 | Cardiac troponin T | ENSSSCG00000023031 |
| TPM1 | α-tropomyosin | NA |
| TTN | Titin | NA |
| VCL | Metavinculin | NA |

*Adapted from L. R. Lopes, P. M. Elliot/Biochimica et Biophysica Acta 1832 (2013) 2451-2461.

As noted, the identified genes are as diverse as RBM20 (RNA Binding Motif 20), a pre-mRNA splicing, scaffold protein that is responsible for high-penetrance of familial DCM and sudden death, presumably due to electrical disturbance [2]; TTN a gene that codes for the structural protein Titin (also known as connectin) that is responsible for connecting the Z line to the M line in muscle sarcomere and is indicated in pathologies of both DCM and HCM; and BAG3 a gene that codes for a protein that is a molecular chaperone for assisted autophagy and is implicated in neurodegenerative diseases in addition to DCM. Other implicated genes include those that code for proteins of muscle sarcomere, cytoskeleton and plasma membrane, desmosomes, the nuclear envelope, transcription/post-transcription regulation and ion-channel/calcium handling (Table 1).

Subsequent investigations of RBM20 has verified a mutation hotspot and establishes this gene as a prototypical cause of DCM with common disease pathways [2-8]. Specifically, confirmatory studies demonstrate that among patients' hearts with end stage HF, several have reduced expression of RBM20 and commensurate RBM20-associated splicing changes for key cardiac genes (e.g. TTN, RYR2, CAMK2D, and LDB3) suggesting that RBM20 drives a common form of genetic HF [6]. Titin, the product of TTN is a giant protein that provides structure, flexibility and stability to muscle proteins such as actin and myosin. Mutations related to TTN are implicated in centronuclear myopathy, familial dilated cardiomyopathy, familial hypertrophic cardiomyopathy, hereditary myopathy with respiratory failure and multiple forms of muscular dystrophy. BAGS is implicated in familial dilated cardiomyopathy and myofibrillar myopathy.

The present disclosure provides genetically engineered animals modeling human mutations in human genes implicated in HF (see, Table 1) will serve as a reproducible, relevant, and reliable model for progression of biventricular HF from children to adults. This first-of-a-kind model enables evidence-based innovation of biologic, pharmaceutical, and device therapies directed towards HF.

Unless defined otherwise, all technical and scientific terms used herein have the same meanings as commonly understood by one of ordinary skill in the art to which this disclosure belongs. All publications and patents specifically mentioned herein are incorporated by reference for all purposes including describing and disclosing the chemicals, instruments, statistical analyses and methodologies which are reported in the publications which might be used in connection with the disclosure. All references cited in this specification are to be taken as indicative of the level of skill in the art. Nothing herein is to be construed as an admission that the disclosure is not entitled to antedate such disclosure by virtue of prior invention.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

“Additive Genetic Effects” as used herein means average individual gene effects that can be transmitted from parent to progeny.

“Allele” as used herein refers to an alternate form of a gene. It also can be thought of as variations of DNA sequence. For instance if an animal has the genotype for a specific gene of Bb, then both B and b are alleles.

As used herein, the term “breaking protein synthesis” refers to any deletion, insertion or mutation that creates a stop codon or frameshift that makes a premature stopping of protein synthesis. Also referred to as a “knockout”.

“DNA Marker” refers to a specific DNA variation that can be tested for association with a physical characteristic.

“Genotype” refers to the genetic makeup of an animal.

As used herein the term “Genomic modification” refers to a modification of the animal genome. A genomically modified animal may have only a single modification of a gene that animal being “genetically modified.” A “genetically modified” animal has a genomic modification.

“Genotyping (DNA marker testing)” refers to the process by which an animal is tested to determine the particular alleles it is carrying for a specific genetic test.

“Simple Traits” refers to traits such as coat color and horned status and some diseases that are carried by a single gene.

“Compound heterozygosity” as used herein is the condition of having two heterogeneous recessive alleles at a particular locus that can cause genetic disease in a heterozygous state.

“Complex Traits” refers to traits such as reproduction, growth and carcass that are controlled by numerous genes.

“Complex allele”-coding region that has more than one mutation within it. This makes it more difficult to determine the effect of a given mutation because researchers cannot be sure which mutation within the allele is causing the effect.

“Copy number variation” (CNVs) a form of structural variation—are alterations of the DNA of a genome that results in the cell having an abnormal or, for certain genes, a normal variation in the number of copies of one or more sections of the DNA. CNVs correspond to relatively large regions of the genome that have been deleted (fewer than the normal number) or duplicated (more than the normal number) on certain chromosomes. For example, the chromosome that normally has sections in order as A-B-C-D might instead have sections A-B-C-“Repetitive element” patterns of nucleic acids (DNA or RNA) that occur in multiple copies throughout the genome. Repetitive DNA was first detected because of its rapid association kinetics.

“Quantitative variation” variation measured on a continuum (e.g. height in human beings) rather than in discrete units or categories. See continuous variation. The existence of a range of phenotypes for a specific character, differing by degree rather than by distinct qualitative differences.

“Homozygous” refers to having two copies of the same allele for a single gene such as BB.

“Heterozygous” refers to having different copies of alleles for a single gene such as Bb.”

“Locus” (plural “loci”) refers to the specific locations of a maker or a gene.

“Centimorgan (Cm)” a unit of recombinant frequency for measuring genetic linkage. It is defined as the distance between chromosome positions (also termed, loci or markers) for which the expected average number of intervening chromosomal crossovers in a single generation is 0.01. It is often used to infer distance along a chromosome. It is not a true physical distance however.

“Chromosomal crossover” (“crossing over”) is the exchange of genetic material between homologous chromosomes inherited by an individual from its mother and father. Each individual has a diploid set (two homologous chromosomes, e.g., 2n) one each inherited from its mother and father. During meiosis I the chromosomes duplicate (4n) and crossover between homologous regions of chromosomes received from the mother and father may occur resulting in new sets of genetic information within each chromosome. Meiosis I is followed by two phases of cell division resulting in four haploid (1n) gametes each carrying a unique set of genetic information. Because genetic recombination results in new gene sequences or combinations of genes, diversity is increased. Crossover usually occurs when homologous regions on homologous chromosomes break and then reconnect to the other chromosome.

“Marker Assisted Selection (MAS)” refers to the process by which DNA marker information is used to assist in making management decisions.

“Marker Panel” a combination of two or more DNA markers that are associated with a particular trait.

“Non-additive Genetic Effects” refers to effects such as dominance and epistasis. Codominance is the interaction of alleles at the same locus while epistasis is the interaction of alleles at different loci.

“Nucleotide” refers to a structural component of DNA that includes one of the four base chemicals: adenine (A), thymine (T), guanine (G), and cytosine (C).

“Phenotype” refers to the outward appearance of an animal that can be measured. Phenotypes are influenced by the genetic makeup of an animal and the environment.

“Single Nucleotide Polymorphism (SNP)” is a single nucleotide change in a DNA sequence.

“Cardiomyopathy” (CM) as used herein refers to diseases of the heart muscle signified by the muscled becoming enlarged, thick or rigid.

“Dilated Cardiomyopathy” (DCM) is signified by the enlargement and weakening of the ventricles.

“Hypertrophic Cardiomyopathy” (HDM) is signified by the enlargement and thickening of the heart muscle.

“Restrictive Cardiomyopathy” (RCM) is signified by the stiffening of the heart muscle.

“Arrhythmogenic Cardiomyopathy” (AVC) disrupts the hearts electrical signals and causes arrhythmias.

As used herein, the term “arrhythmia” refers to heart arrhythmias, also known as cardiac dysrhythmia or irregular heartbeat, is a group of conditions in which the heartbeat is irregular, too fast, or too slow. There are four main types of arrhythmia: extra beats, supraventricular tachycardias, ventricular arrhythmias, and bradyarrhythmias. Extra beats include premature atrial contractions, premature ventricular contractions, and premature junctional contractions. Supraventricular tachycardias include atrial fibrillation, atrial flutter, and paroxysmal supraventricular tachycardia. Ventricular arrhythmias include ventricular fibrillation and ventricular tachycardia. Arrhythmias are due to problems with the electrical conduction system of the heart. Arrhythmias may occur in children; however, the normal range for the heart rate is different and depends on age. A number of tests can help with diagnosis including an electrocardiogram (ECG/EKG) and Holter monitor.

As used herein, the term “cardiac function” refers to measurements used to determine the wellness of cardiac functioning. Such measurements include: left ventricular ejection fraction (LVEF), expressed as the ratio of the left ventricular stroke volume (SV) to the left ventricular end-diastolic volume (LVEDV). SV is obtained by subtracting the left ventricular end-systolic volume (LVESV) from LVEDV. Plasma NT-BNP refers to the circulating levels of N-Terminal fragment of brain natriuretic peptide which activates atrial natriuretic peptide receptor (NPRB). BNP acts to decrease systemic vascular resistance, central venous pressure and increase natriuresis. Those of skill in the art recognize that the function of the right heart (e.g., right atrium/right ventricle) can be measured similarly.

“Haploid genotype” or “haplotype” refers to a combination of alleles, loci or DNA polymorphisms that are linked so as to cosegregate in a significant proportion of gametes during meiosis. The alleles of a haplotype may be in linkage disequilibrium (LD).

“Linkage disequilibrium (LD)” is the non-random association of alleles at different loci i.e. the presence of statistical associations between alleles at different loci that are different from what would be expected if alleles were independently, randomly sampled based on their individual allele frequencies. If there is no linkage disequilibrium between alleles at different loci they are said to be in linkage equilibrium.

The term “restriction fragment length polymorphism” or “RFLP” refers to any one of different DNA fragment lengths produced by restriction digestion of genomic DNA or cDNA with one or more endonuclease enzymes, wherein the fragment length varies between individuals in a population.

“Introgression” also known as “introgressive hybridization”, is the movement of a gene or allele (gene flow) from one species into the gene pool of another by the repeated backcrossing of an interspecific hybrid with one of its parent species. Purposeful introgression is a long-term process; it may take many hybrid generations before the backcrossing occurs.

“Nonmeiotic introgression” genetic introgression via introduction of a gene or allele in a diploid (non-gemetic) cell. Non-meiotic introgression does not rely on sexual reproduction and does not require backcrossing and, significantly, is carried out in a single generation. In non-meiotic introgression an allele is introduced into a haplotype via homologous recombination. The allele may be introduced at the site of an existing allele to be edited from the genome or the allele can be introduced at any other desirable site.

As used herein the term “genetic modification” refers to is the direct manipulation of an organism genome using biotechnology.

As used herein the phrase “precision gene editing” or “gene editing” means a process gene modification which allows geneticists to introduce (introgress) any natural trait into any breed, in a site-specific manner without the use of recombinant DNA. Gene edited animals are not transgenic and do

As used herein the phrase “multiplex gene editing” refers to the editing of multiple genes during a single editing event. In these instances, multiple specific editing nucleases target different genes or loci.

“Transcription activator-like effector nucleases (TALENs)” one technology for gene editing are artificial restriction enzymes generated by fusing a TAL effector DNA-binding domain to a DNA cleavage domain.

“Zinc finger nucleases (ZFNs)” as used herein are another technology useful for gene editing and are a class of engineered DNA-binding proteins that facilitate targeted editing of the genome by creating double-strand breaks in DNA at user-specified locations.

“Meganuclease” as used herein are another technology useful for gene editing and are endodeoxyribonucleases characterized by a large recognition site (double-stranded DNA sequences of 12 to 40 base pairs); as a result this site generally occurs only once in any given genome. For example, the 18-base pair sequence recognized by the I-SceI meganuclease would on average require a genome twenty times the size of the human genome to be found once by chance (although sequences with a single mismatch occur about three times per human-sized genome). Meganucleases are therefore considered to be the most specific naturally occurring restriction enzymes.

“CRISPR/CAS” as used herein, refers another gene editing technology “CRISPRs” (clustered regularly interspaced short palindromic repeats), segments of prokaryotic DNA containing short repetitions of base sequences. Each repetition is followed by short segments of “spacer DNA” from previous exposures to a bacterial virus or plasmid. “CAS” (CRISPR associated protein 9) is an RNA-guided DNA endonuclease enzyme associated with the CRISPR. By delivering the Cas9 protein and appropriate guide RNAs into a cell, the organism's genome can be cut at any desired location.

“Indel” as used herein is shorthand for “insertion” or “deletion” referring to a modification of the DNA in an organism.

As used herein the term “renucleated egg” refers to an enucleated egg used for somatic cell nuclear transfer in which the modified nucleus of a somatic cell has been introduced.

“Genetic marker” as used herein refers to a gene/allele or known DNA sequence with a known location on a chromosome. The markers may be any genetic marker e.g., one or more alleles, haplotypes, haplogroups, loci, quantitative trait loci, or DNA polymorphisms [restriction fragment length polymorphisms (RFLPs), amplified fragment length polymorphisms (AFLPs), single nuclear polymorphisms (SNPs), indels, short tandem repeats (STRs), microsatellites and minisatellites]. Conveniently, the markers are SNPs or STRs such as microsatellites, and more preferably SNPs. Preferably, the markers within each chromosome segment are in linkage disequilibrium.

As used herein the term “host animal” means an animal which has a native genetic complement of a recognized species or breed of animal.

As used herein, “native haplotype” or “native genome” means the natural DNA of a particular species or breed of animal that is chosen to be the recipient of a gene or allele that is not present in the host animal.

As used herein the term “target” means a specific thing to which some other entity (e.g., an endogenous ligand (nuclease) or a drug i) is directed to or binds to.

As used herein the term “target locus” means a specific location on a chromosome.

As used herein the term “target site” means the specific location targeted by an entity.

As used herein the term “site specific” means created or designed for a specific site with no cross reaction or binding with other sites or locations.

As used, herein, the term “quantitative trait” refers to a trait that fits into discrete categories. Quantitative traits occur as a continuous range of variation such as that amount of milk a particular breed can give or the length of a tail. Generally, a larger group of genes controls quantitative traits.

As used herein, the term “qualitative trait” is used to refer to a trait that falls into different categories. These categories do not have any certain order. As a general rule, qualitative traits are monogenic, meaning the trait is influenced by a single gene. Examples of qualitative traits include blood type and flower color, for example.

As used herein, the term “quantitative trait locus (QTL)” is a section of DNA (the locus) that correlates with variation in a phenotype (the quantitative trait).

As used herein the term “cloning” means production of genetically identical organisms asexually.

“Somatic cell nuclear transfer” (“SCNT”) is one strategy for cloning a viable embryo from a body cell and an egg cell. The technique consists of taking an enucleated oocyte (egg cell) and implanting a donor nucleus from a somatic (body) cell.

“Orthologous” as used herein refers to a gene with similar function to a gene in an evolutionarily related species. The identification of orthologues is useful for gene function prediction. In the case of livestock, orthologous genes are found throughout the animal kingdom and those found in other mammals may be particularly useful for transgenic replacement. This is particularly true for animals of the same species, breed or lineages wherein species are defined two animals so closely related as to being able to produce fertile offspring via sexual reproduction; breed is defined as a specific group of domestic animals having homogenous phenotype, homogenous behavior and other characteristics that define the animal from others of the same species; and wherein lineage is defined as continuous line of descent; a series of organisms, populations, cells, or genes connected by ancestor/descendent relationships. For example domesticated cattle are of two distinct lineages both arising from ancient aurochs. One lineage descends from the domestication of aurochs in the Middle East while the second distinct lineage descends from the domestication of the aurochs on the Indian subcontinent.

“Genotyping” or “genetic testing” generally refers to detecting one or more markers of interest e.g., SNPs in a sample from an individual being tested, and analyzing the results obtained to determine the haplotype of the subject. As will be apparent from the disclosure herein, it is one exemplary embodiment to detect the one or more markers of interest using a high-throughput system comprising a solid support consisting essentially of or having nucleic acids of different sequence bound directly or indirectly thereto, wherein each nucleic acid of different sequence comprises a polymorphic genetic marker derived from an ancestor or founder that is representative of the current population and, more preferably wherein said high-throughput system comprises sufficient markers to be representative of the genome of the current population. Preferred samples for genotyping comprise nucleic acid, e.g., RNA or genomic DNA and preferably genomic DNA. A breed of livestock animal can be readily established by evaluating its genetic markers.

The term “proximate” as used herein means close to.

Livestock may be genotyped to identify various genetic markers. Genotyping is a term that refers to the process of determining differences in the genetic make-up (genotype) of an individual by determining the individual's DNA sequence using a biological assay and comparing it to another individual's sequence or to a reference sequence. A genetic marker is a known DNA sequence, with a known location on a chromosome; they are consistently passed on through breeding, so they can be traced through a pedigree or phylogeny. Genetic markers can be a sequence comprising a plurality of bases, or a single nucleotide polymorphism (SNP) at a known location. The breed of a livestock animal can be readily established by evaluating its genetic markers. Many markers are known and there are many different measurement techniques that attempt to correlate the markers to traits of interest, or to establish a genetic value of an animal for purposes of future breeding or expected value.

Homology directed repair (HDR) is a mechanism in cells to repair ssDNA and double stranded DNA (dsDNA) lesions. This repair mechanism can be used by the cell when there is an HDR template present that has a sequence with significant homology to the lesion site. Specific binding, as that term is commonly used in the biological arts, refers to a molecule that binds to a target with a relatively high affinity compared to non-target tissues, and generally involves a plurality of non-covalent interactions, such as electrostatic interactions, van der Waals interactions, hydrogen bonding, and the like. Specific hybridization is a form of specific binding between nucleic acids that have complementary sequences. Proteins can also specifically bind to DNA, for instance, in TALENs or CRISPR/Cas9 systems or by Gal4 motifs. Introgression of an allele refers to a process of copying an exogenous allele over an endogenous allele with a template-guided process. The endogenous allele might actually be excised and replaced by an exogenous nucleic acid allele in some situations but present theory is that the process is a copying mechanism. Since alleles are gene pairs, there is significant homology between them. The allele might be a gene that encodes a protein, or it could have other functions such as encoding a bioactive RNA chain or providing a site for receiving a regulatory protein or RNA.

The HDR template is a nucleic acid that comprises the allele that is being introgressed. The template may be a dsDNA or a single-stranded DNA (ssDNA). ssDNA templates are preferably from about 20 to about 5000 residues although other lengths can be used. Artisans will immediately appreciate that all ranges and values within the explicitly stated range are contemplated; e.g., from 500 to 1500 residues, from 20 to 100 residues, and so forth. The template may further comprise flanking sequences that provide homology to DNA adjacent to the endogenous allele or the DNA that is to be replaced. The template may also comprise a sequence that is bound to a targeted nuclease system, and is thus the cognate binding site for the system's DNA-binding member. The term cognate refers to two biomolecules that typically interact, for example, a receptor and its ligand. In the context of HDR processes, one of the biomolecules may be designed with a sequence to bind with an intended, i.e., cognate, DNA site or protein site.

### Targeted Endonuclease Systems

Genome editing tools such as transcription activator-like effector nucleases (TALENs) and zinc finger nucleases (ZFNs) have impacted the fields of biotechnology, gene therapy and functional genomic studies in many organisms. More recently, RNA-guided endonucleases (RGENs) are directed to their target sites by a complementary RNA molecule. The Cas9/CRISPR system is a REGEN. TracrRNA is another such tool. These are examples of targeted nuclease systems: these system have a DNA-binding member that localizes the nuclease to a target site. The site is then cut by the nuclease. TALENs and ZFNs have the nuclease fused to the DNA-binding member. Cas9/CRISPR are cognates that find each other on the target DNA. The DNA-binding member has a cognate sequence in the chromosomal DNA. The DNA-binding member is typically designed in light of the intended cognate sequence so as to obtain a nucleolytic action at nor near an intended site. Certain embodiments are applicable to all such systems without limitation; including, embodiments that minimize nuclease re-cleavage, embodiments for making SNPs with precision at an intended residue, and placement of the allele that is being introgressed at the DNA-binding site.

### TALENs

The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA or a TALEN-pair.

The cipher for TALs has been reported (PCT Publication WO 2011/072246) wherein each DNA binding repeat is responsible for recognizing one base pair in the target DNA sequence. The residues may be assembled to target a DNA sequence. In brief, a target site for binding of a TALEN is determined and a fusion molecule comprising a nuclease and a series of RVDs that recognize the target site is created. Upon binding, the nuclease cleaves the DNA so that cellular repair machinery can operate to make a genetic modification at the cut ends. The term TALEN means a protein comprising a Transcription Activator-like (TAL) effector binding domain and a nuclease domain and includes monomeric TALENs that are functional per se as well as others that require dimerization with another monomeric TALEN. The dimerization can result in a homodimeric TALEN when both monomeric TALEN are identical or can result in a heterodimeric TALEN when monomeric TALEN are different. TALENs have been shown to induce gene modification in immortalized human cells by means of the two major eukaryotic DNA repair pathways, non-homologous end joining (NHEJ) and homology directed repair. TALENs are often used in pairs but monomeric TALENs are known. Cells for treatment by TALENs (and other genetic tools) include a cultured cell, an immortalized cell, a primary cell, a primary somatic cell, a zygote, a germ cell, a primordial germ cell, a blastocyst, or a stem cell. In some embodiments, a TAL effector can be used to target other protein domains (e.g., non-nuclease protein domains) to specific nucleotide sequences. For example, a TAL effector can be linked to a protein domain from, without limitation, a DNA 20 interacting enzyme (e.g., a methylase, a topoisomerase, an integrase, a transposase, or a ligase), a transcription activators or repressor, or a protein that interacts with or modifies other proteins such as histones. Applications of such TAL effector fusions include, for example, creating or modifying epigenetic regulatory elements, making site-specific insertions, deletions, or repairs in DNA, controlling gene expression, and modifying chromatin structure.

The term nuclease includes exonucleases and endonucleases. The term endonuclease refers to any wild-type or variant enzyme capable of catalyzing the hydrolysis (cleavage) of bonds between nucleic acids within a DNA or RNA molecule, preferably a DNA molecule. Non-limiting examples of endonucleases include type II restriction endonucleases such as FokI, HhaI, HindIII, NotI, BbvCl, EcoRI, BglII, and AlwI. Endonucleases comprise also rare-cutting endonucleases when having typically a polynucleotide recognition site of about 12-45 basepairs (bp) in length, more preferably of 14-45 bp. Rare-cutting endonucleases induce DNA double-strand breaks (DSBs) at a defined locus. Rare-cutting endonucleases can for example be a targeted endonuclease, a chimeric Zinc-Finger nuclease (ZFN) resulting from the fusion of engineered zinc-finger domains with the catalytic domain of a restriction enzyme such as FokI or a chemical endonuclease. In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences. Such chemical endonucleases are comprised in the term “endonuclease” according to the present invention. Examples of such endonuclease include I-See I, I-Chu L I-Cre I, I-Csm I, PI-See L PI-Tti L PI-Mtu I, I-Ceu I, I-See IL I-See III, HO, PI-Civ I, PI-Ctr L PI-Aae I, PI-Bsu I, PI-Dha I, PI-Dra L PI-May L PI-Meh I, PI-Mfu L PI-Mfl I, PI-Mga L PI-Mgo I, PI-Min L PI-Mka L PI-Mle I, PI-Mma I, PI-30 Msh L PI-Msm I, PI-Mth I, PI-Mtu I, PI-Mxe I, PI-Npu I, PI-Pfu L PI-Rma I, PI-Spb I, PI-Ssp L PI-Fae L PI-Mja I, PI-Pho L PI-Tag L PI-Thy I, PI-Tko I, PI-Tsp I, I-Msol.

A genetic modification made by TALENs or other tools may be, for example, chosen from the list consisting of an insertion, a deletion, insertion of an exogenous nucleic acid fragment, and a substitution. The term insertion is used broadly to mean either literal insertion into the chromosome or use of the exogenous sequence as a template for repair. In general, a target DNA site is identified and a TALEN-pair is created that will specifically bind to the site. The TALEN is delivered to the cell or embryo, e.g., as a protein, mRNA or by a vector that encodes the TALEN. The TALEN cleaves the DNA to make a double-strand break that is then repaired, often resulting in the creation of an indel, or incorporating sequences or polymorphisms contained in an accompanying exogenous nucleic acid that is either inserted into the chromosome or serves as a template for repair of the break with a modified sequence. This template-driven repair is a useful process for changing a chromosome, and provides for effective changes to cellular chromosomes.

The term exogenous nucleic acid means a nucleic acid that is added to the cell or embryo, regardless of whether the nucleic acid is the same or distinct from nucleic acid sequences naturally in the cell. The term nucleic acid fragment is broad and includes a chromosome, expression cassette, gene, DNA, RNA, mRNA, or portion thereof. The cell or embryo may be, for instance, chosen from the group consisting non-human vertebrates, non-human primates, cattle, horse, swine, sheep, chicken, avian, rabbit, goats, dog, cat, laboratory animal, and fish.

Some embodiments involve a composition or a method of making a genetically modified livestock and/or artiodactyl comprising introducing a TALEN-pair into livestock and/or an artiodactyl cell or embryo that makes a genetic modification to DNA of the cell or embryo at a site that is specifically bound by the TALEN-pair, and producing the livestock animal/artiodactyl from the cell. Direct injection may be used for the cell or embryo, e.g., into a zygote, blastocyst, or embryo. Alternatively, the TALEN and/or other factors may be introduced into a cell using any of many known techniques for introduction of proteins, RNA, mRNA, DNA, or vectors. Genetically modified animals may be made from the embryos or cells according to known processes, e.g., implantation of the embryo into a gestational host, or various cloning methods. The phrase “a genetic modification to DNA of the cell at a site that is specifically bound by the TALEN”, or the like, means that the genetic modification is made at the site cut by the nuclease on the TALEN when the TALEN is specifically bound to its target site. The nuclease does not cut exactly where the TALEN-pair binds, but rather at a defined site between the two binding sites.

Some embodiments involve a composition or a treatment of a cell that is used for cloning the animal. The cell may be a livestock and/or artiodactyl cell, a cultured cell, a primary cell, a primary somatic cell, a zygote, a germ cell, a primordial germ cell, or a stem cell. For example, an embodiment is a composition or a method of creating a genetic modification comprising exposing a plurality of primary cells in a culture to TALEN proteins or a nucleic acid encoding a TALEN or TALENs. The TALENs may be introduced as proteins or as nucleic acid fragments, e.g., encoded by mRNA or a DNA sequence in a vector.

### Zinc Finger Nucleases

Zinc-finger nucleases (ZFNs) are artificial restriction enzymes generated by fusing a zinc finger DNA-binding domain to a DNA-cleavage domain. Zinc finger domains can be engineered to target desired DNA sequences and this enables zinc-finger nucleases to target unique sequences within complex genomes. By taking advantage of endogenous DNA repair machinery, these reagents can be used to alter the genomes of higher organisms. ZFNs may be used in method of inactivating genes.

A zinc finger DNA-binding domain has about 30 amino acids and folds into a stable structure. Each finger primarily binds to a triplet within the DNA substrate. Amino acid residues at key positions contribute to most of the sequence-specific interactions with the DNA site. These amino acids can be changed while maintaining the remaining amino acids to preserve the necessary structure. Binding to longer DNA sequences is achieved by linking several domains in tandem. Other functionalities like non-specific FokI cleavage domain (N), transcription activator domains (A), transcription repressor domains (R) and methylases (M) can be fused to a ZFPs to form ZFNs respectively, zinc finger transcription activators (ZFA), zinc finger transcription repressors (ZFR, and zinc finger methylases (ZFM). Materials and methods for using zinc fingers and zinc finger nucleases for making genetically modified animals are disclosed in, e.g., U.S. Pat. No. 8,106,255; U.S. 2012/0192298; U.S. 2011/0023159; and U.S. 2011/0281306.

### Vectors and Nucleic Acids

A variety of nucleic acids may be introduced into cells, for knockout purposes, for inactivation of a gene, to obtain expression of a gene, or for other purposes. As used herein, the term nucleic acid includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained.

The target nucleic acid sequence can be operably linked to a regulatory region such as a promoter. Regulatory regions can be porcine regulatory regions or can be from other species. As used herein, operably linked refers to positioning of a regulatory region relative to a nucleic acid sequence in such a way as to permit or facilitate transcription of the target nucleic acid.

In general, type of promoter can be operably linked to a target nucleic acid sequence. Examples of promoters include, without limitation, tissue-specific promoters, constitutive promoters, inducible promoters, and promoters responsive or unresponsive to a particular stimulus. In some embodiments, a promoter that facilitates the expression of a nucleic acid molecule without significant tissue- or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. In some embodiments, a fusion of the chicken beta actin gene promoter and the CMV enhancer is used as a promoter. See, for example, Xu et al., Hum. Gene Ther. 12:563, 2001; and Kiwaki et al., Hum. Gene Ther. 7:821, 1996.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

A nucleic acid construct may be used that encodes signal peptides or selectable expressed markers. Signal peptides can be used such that an encoded polypeptide is directed to a particular cellular location (e.g., the cell surface). Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

In some embodiments, a sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban et al., Proc. Natl. Acad. Sci., 89:6861, 1992, for a review of Cre/lox technology, and Brand and Dymecki, Dev. Cell, 6:7, 2004. A transposon containing a Cre- or Flp-activatable transgene interrupted by a selectable marker gene also can be used to obtain transgenic animals with conditional expression of a transgene. For example, a promoter driving expression of the marker/transgene can be either ubiquitous or tissue-specific, which would result in the ubiquitous or tissue-specific expression of the marker in F0 animals (e.g., pigs). Tissue specific activation of the transgene can be accomplished, for example, by crossing a pig that ubiquitously expresses a marker-interrupted transgene to a pig expressing Cre or Flp in a tissue-specific manner, or by crossing a pig that expresses a marker-interrupted transgene in a tissue-specific manner to a pig that ubiquitously expresses Cre or Flp recombinase. Controlled expression of the transgene or controlled excision of the marker allows expression of the transgene.

In some embodiments, the exogenous nucleic acid encodes a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST) and FLAG™ tag (Kodak, New Haven, Conn.).

Nucleic acid constructs can be introduced into embryonic, fetal, or adult artiodactyl/livestock cells of any type, including, for example, germ cells such as an oocyte or an egg, a progenitor cell, an adult or embryonic stem cell, a primordial germ cell, a kidney cell such as a PK-15 cell, an islet cell, a beta cell, a liver cell, or a fibroblast such as a dermal fibroblast, using a variety of techniques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells.

In transposon systems, the transcriptional unit of a nucleic acid construct, i.e., the regulatory region operably linked to an exogenous nucleic acid sequence, is flanked by an inverted repeat of a transposon. Several transposon systems, including, for example, Sleeping Beauty (see, U.S. Pat. No. 6,613,752 and U.S. 2005/0003542); Frog Prince (Miskey et al., Nucleic Acids Res., 31:6873, 2003); Tol2 (Kawakami, Genome Biology, 8(Supp1.1):S7, 2007); Minos (Pavlopoulos et al., Genome Biology, 8(Supp1.1):S2, 2007); Hsmarl (Miskey et al., Mol Cell Biol., 27:4589, 2007); and Passport have been developed to introduce nucleic acids into cells, including mice, human, and pig cells. The Sleeping Beauty transposon is particularly useful. A transposase can be delivered as a protein, encoded on the same nucleic acid construct as the exogenous nucleic acid, can be introduced on a separate nucleic acid construct, or provided as an mRNA (e.g., an in vitro-transcribed and capped mRNA).

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in animals have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, and also any necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., modified HIV-1, SIV or FIV), retrovirus (e.g., ASV, ALV or MoMLV), and transposons (e.g., Sleeping Beauty, P-elements, Tol-2, Frog Prince, piggyBac).

As used herein, the term nucleic acid refers to both RNA and DNA, including, for example, cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand). The term transgenic is used broadly herein and refers to a genetically modified organism or genetically engineered organism whose genetic material has been altered using genetic engineering techniques. A knockout artiodactyl is thus transgenic regardless of whether or not exogenous genes or nucleic acids are expressed in the animal or its progeny.

### Genetically Modified Animals

Animals may be modified using TALENs or other genetic engineering tools, including recombinase fusion proteins, or various vectors that are known. A genetic modification made by such tools may comprise disruption of a gene. The term disruption of a gene refers to preventing the formation of a functional gene product. A gene product is functional only if it fulfills its normal (wild-type) functions. Disruption of the gene prevents expression of a functional factor encoded by the gene and comprises an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene. Materials and methods of genetically modifying animals are further detailed in U.S. Pat. No. 8,518,701; U.S. 2010/0251395; and U.S. 2012/0222143 which are hereby incorporated herein by reference for all purposes; in case of conflict, the instant specification is controlling. The term trans-acting refers to processes acting on a target gene from a different molecule (i.e., intermolecular). A trans-acting element is usually a DNA sequence that contains a gene. This gene codes for a protein (or microRNA or other diffusible molecule) that is used in the regulation the target gene. The trans-acting gene may be on the same chromosome as the target gene, but the activity is via the intermediary protein or RNA that it encodes. Embodiments of trans-acting gene are, e.g., genes that encode targeting endonucleases. Inactivation of a gene using a dominant negative generally involves a trans-acting element. The term cis-regulatory or cis-acting means an action without coding for protein or RNA; in the context of gene inactivation, this generally means inactivation of the coding portion of a gene, or a promoter and/or operator that is necessary for expression of the functional gene.

Various techniques known in the art can be used to inactivate genes to make knock-out animals and/or to introduce nucleic acid constructs into animals to produce founder animals and to make animal lines, in which the knockout or nucleic acid construct is integrated into the genome. Such techniques include, without limitation, pronuclear microinjection (U.S. Pat. No. 4,873,191), retrovirus mediated gene transfer into germ lines (Van der Putten et al., Proc. Natl. Acad. Sci. USA, 82:6148-6152, 1985), gene targeting into embryonic stem cells (Thompson et al., Cell, 56:313-321, 1989), electroporation of embryos (Lo, Mol. Cell. Biol., 3:1803-1814, 1983), sperm-mediated gene transfer (Lavitrano et al., Proc. Natl. Acad. Sci. USA, 99:14230-14235, 2002; Lavitrano et al., Reprod. Fert. Develop., 18:19-23, 2006), and in vitro transformation of somatic cells, such as cumulus or mammary cells, or adult, fetal, or embryonic stem cells, followed by nuclear transplantation (Wilmut et al., Nature, 385:810-813, 1997; and Wakayama et al., Nature, 394:369-374, 1998). Pronuclear microinjection, sperm mediated gene transfer, and somatic cell nuclear transfer are particularly useful techniques. An animal that is genomically modified is an animal wherein all of its cells have the genetic modification, including its germ line cells. When methods are used that produce an animal that is mosaic in its genetic modification, the animals may be inbred and progeny that are genomically modified may be selected. Cloning, for instance, may be used to make a mosaic animal if its cells are modified at the blastocyst state, or genomic modification can take place when a single-cell is modified. Animals that are modified so they do not sexually mature can be homozygous or heterozygous for the modification, depending on the specific approach that is used. If a particular gene is inactivated by a knock out modification, homozygosity would normally be required. If a particular gene is inactivated by an RNA interference or dominant negative strategy, then heterozygosity is often adequate.

Typically, in pronuclear microinjection, a nucleic acid construct is introduced into a fertilized egg; 1 or 2 cell fertilized eggs are used as the pronuclei containing the genetic material from the sperm head and the egg are visible within the protoplasm. Pronuclear staged fertilized eggs can be obtained in vitro or in vivo (i.e., surgically recovered from the oviduct of donor animals). In vitro fertilized eggs can be produced as follows. For example, swine ovaries can be collected at an abattoir, and maintained at 22-28° C. during transport. Ovaries can be washed and isolated for follicular aspiration, and follicles ranging from 4-8 mm can be aspirated into 50 mL conical centrifuge tubes using 18 gauge needles and under vacuum. Follicular fluid and aspirated oocytes can be rinsed through pre-filters with commercial TL-HEPES (Minitube, Verona, Wis.). Oocytes surrounded by a compact cumulus mass can be selected and placed into TCM-199 OOCYTE MATURATION MEDIUM (Minitube, Verona, Wis.) supplemented with 0.1 mg/mL cysteine, 10 ng/mL epidermal growth factor, 10% porcine follicular fluid, 50 μM 2-mercaptoethanol, 0.5 mg/ml cAMP, 10 IU/mL each of pregnant mare serum gonadotropin (PMSG) and human chorionic gonadotropin (hCG) for approximately 22 hours in humidified air at 38.7° C. and 5% CO₂. Subsequently, the oocytes can be moved to fresh TCM-199 maturation medium, which will not contain cAMP, PMSG or hCG and incubated for an additional 22 hours. Matured oocytes can be stripped of their cumulus cells by vortexing in 0.1% hyaluronidase for 1 minute. For swine, mature oocytes can be fertilized in 500 μl Minitube PORCPRO IVF MEDIUM SYSTEM (Minitube, Verona, Wis.) in Minitube 5-well fertilization dishes. In preparation for in vitro fertilization (IVF), freshly-collected or frozen boar semen can be washed and resuspended in PORCPRO IVF Medium to 4×10⁵ sperm. Sperm concentrations can be analyzed by computer assisted semen analysis (SPERMVISION, Minitube, Verona, Wis.). Final in vitro insemination can be performed in a 10 μl volume at a final concentration of approximately 40 motile sperm/oocyte, depending on boar. Incubate all fertilizing oocytes at 38.7° C. in 5.0% CO₂ atmosphere for 6 hours. Six hours post-insemination, presumptive zygotes can be washed twice in NCSU-23 and moved to 0.5 mL of the same medium. This system can produce 20-30% blastocysts routinely across most boars with a 10-30% polyspermic insemination rate.

Linearized nucleic acid constructs can be injected into one of the pronuclei. Then the injected eggs can be transferred to a recipient female (e.g., into the oviducts of a recipient female) and allowed to develop in the recipient female to produce the transgenic animals. In particular, in vitro fertilized embryos can be centrifuged at 15,000×g for 5 minutes to sediment lipids allowing visualization of the pronucleus. The embryos can be injected with using an Eppendorf FEMTOJET injector and can be cultured until blastocyst formation. Rates of embryo cleavage and blastocyst formation and quality can be recorded.

Embryos can be surgically transferred into uteri of asynchronous recipients. Typically, 100-200 (e.g., 150-200) embryos can be deposited into the ampulla-isthmus junction of the oviduct using a 5.5-inch TOMCAT® catheter. After surgery, real-time ultrasound examination of pregnancy can be performed.

In somatic cell nuclear transfer, a transgenic artiodactyl cell (e.g., a transgenic pig cell or bovine cell) such as an embryonic blastomere, fetal fibroblast, adult ear fibroblast, or granulosa cell that includes a nucleic acid construct described above, can be introduced into an enucleated oocyte to establish a combined cell. Oocytes can be enucleated by partial zona dissection near the polar body and then pressing out cytoplasm at the dissection area. Typically, an injection pipette with a sharp beveled tip is used to inject the transgenic cell into an enucleated oocyte arrested at meiosis 2. In some conventions, oocytes arrested at meiosis-2 are termed eggs. After producing a porcine or bovine embryo (e.g., by fusing and activating the oocyte), the embryo is transferred to the oviducts of a recipient female, about 20 to 24 hours after activation. See, for example, Cibelli et al., Science, 280:1256-1258, 1998; and U.S. Pat. No. 6,548,741. For pigs, recipient females can be checked for pregnancy approximately 20-21 days after transfer of the embryos.

Standard breeding techniques can be used to create animals that are homozygous for the exogenous nucleic acid from the initial heterozygous founder animals. Homozygosity may not be required, however. Transgenic pigs described herein can be bred with other pigs of interest.

In some embodiments, a nucleic acid of interest and a selectable marker can be provided on separate transposons and provided to either embryos or cells in unequal amount, where the amount of transposon containing the selectable marker far exceeds (5-10 fold excess) the transposon containing the nucleic acid of interest. Transgenic cells or animals expressing the nucleic acid of interest can be isolated based on presence and expression of the selectable marker. Because the transposons will integrate into the genome in a precise and unlinked way (independent transposition events), the nucleic acid of interest and the selectable marker are not genetically linked and can easily be separated by genetic segregation through standard breeding. Thus, transgenic animals can be produced that are not constrained to retain selectable markers in subsequent generations, an issue of some concern from a public safety perspective.

Once transgenic animal have been generated, expression of an exogenous nucleic acid can be assessed using standard techniques. Initial screening can be accomplished by Southern blot analysis to determine whether or not integration of the construct has taken place. For a description of Southern analysis, see sections 9.37-9.52 of Sambrook et al., Molecular Cloning, A Laboratory Manual, second edition, Cold Spring Harbor Press, Plainview; NY., 1989. Polymerase chain reaction (PCR) techniques also can be used in the initial screening. PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. PCR is described in, for example PCR Primer: A Laboratory Manual, ed. Dieffenbach and Dveksler, Cold Spring Harbor Laboratory Press, 1995. Nucleic acids also can be amplified by ligase chain reaction, strand displacement amplification, self-sustained sequence replication, or nucleic acid sequence-based amplified. See, for example, Lewis, Genetic Engineering News, 12:1, 1992; Guatelli et al., Proc. Natl. Acad. Sci. USA, 87:1874, 1990; and Weiss, Science, 254:1292, 1991. At the blastocyst stage, embryos can be individually processed for analysis by PCR, Southern hybridization and splinkerette PCR (see, e.g., Dupuy et al., Proc Natl Acad Sci USA, 99:4495, 2002).

Expression of a nucleic acid sequence encoding a polypeptide in the tissues of transgenic pigs can be assessed using techniques that include, for example, Northern blot analysis of tissue samples obtained from the animal, in situ hybridization analysis, Western analysis, immunoassays such as enzyme-linked immunosorbent assays, and reverse-transcriptase PCR (RT-PCR).

### Interfering RNAs

A variety of interfering RNA (RNAi) are known. Double-stranded RNA (dsRNA) induces sequence-specific degradation of homologous gene transcripts. RNA-induced silencing complex (RISC) metabolizes dsRNA to small 21-23-nucleotide small interfering RNAs (siRNAs). RISC contains a double stranded RNAse (dsRNase, e.g., Dicer) and ssRNase (e.g., Argonaut 2 or Ago2). RISC utilizes antisense strand as a guide to find a cleavable target. Both siRNAs and microRNAs (miRNAs) are known. A method of disrupting a gene in a genetically modified animal comprises inducing RNA interference against a target gene and/or nucleic acid such that expression of the target gene and/or nucleic acid is reduced.

For example the exogenous nucleic acid sequence can induce RNA interference against a nucleic acid encoding a polypeptide. For example, double-stranded small interfering RNA (siRNA) or small hairpin RNA (shRNA) homologous to a target DNA can be used to reduce expression of that DNA. Constructs for siRNA can be produced as described, for example, in Fire et al., Nature, 391:806, 1998; Romano and Masino, Mol. Microbiol., 6:3343, 1992; Cogoni et al., EMBO J., 15:3153, 1996; Cogoni and Masino, Nature, 399:166, 1999; Misquitta and Paterson Proc. Natl. Acad. Sci. USA, 96:1451, 1999; and Kennerdell and Carthew, Cell, 95:1017, 1998. Constructs for shRNA can be produced as described by McIntyre and Fanning (2006) BMC Biotechnology 6:1. In general, shRNAs are transcribed as a single-stranded RNA molecule containing complementary regions, which can anneal and form short hairpins.

The probability of finding a single, individual functional siRNA or miRNA directed to a specific gene is high. The predictability of a specific sequence of siRNA, for instance, is about 50% but a number of interfering RNAs may be made with good confidence that at least one of them will be effective.

Embodiments include an in vitro cell, an in vivo cell, and a genetically modified animal such as a livestock animal that express an RNAi directed against a gene, e.g., a gene selective for a developmental stage. The RNAi may be, for instance, selected from the group consisting of siRNA, shRNA, dsRNA, RISC and miRNA.

### Inducible Systems

An inducible system may be used to control expression of a gene. Various inducible systems are known that allow spatiotemporal control of expression of a gene. Several have been proven to be functional in vivo in transgenic animals. The term inducible system includes traditional promoters and inducible gene expression elements.

An example of an inducible system is the tetracycline (tet)-on promoter system, which can be used to regulate transcription of the nucleic acid. In this system, a mutated Tet repressor (TetR) is fused to the activation domain of herpes simplex virus VP16 trans-activator protein to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the animal to trigger the inducible system is referred to as an induction agent.

The tetracycline-inducible system and the Cre/loxP recombinase system (either constitutive or inducible) are among the more commonly used inducible systems. The tetracycline-inducible system involves a tetracycline-controlled transactivator (tTA)/reverse tTA (rtTA). A method to use these systems in vivo involves generating two lines of genetically modified animals. One animal line expresses the activator (tTA, rtTA, or Cre recombinase) under the control of a selected promoter. Another set of transgenic animals express the acceptor, in which the expression of the gene of interest (or the gene to be modified) is under the control of the target sequence for the tTA/rtTA transactivators (or is flanked by loxP sequences). Mating the two strains of mice provides control of gene expression.

The tetracycline-dependent regulatory systems (tet systems) rely on two components, i.e., a tetracycline-controlled transactivator (tTA or rtTA) and a tTA/rtTA-dependent promoter that controls expression of a downstream cDNA, in a tetracycline-dependent manner. In the absence of tetracycline or its derivatives (such as doxycycline), tTA binds to tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. Administration of tetracycline or its derivatives allows temporal control of transgene expression in vivo. rtTA is a variant of tTA that is not functional in the absence of doxycycline but requires the presence of the ligand for transactivation. This tet system is therefore termed tet-ON. The tet systems have been used in vivo for the inducible expression of several transgenes, encoding, e.g., reporter genes, oncogenes, or proteins involved in a signaling cascade.

The Cre/lox system uses the Cre recombinase, which catalyzes site-specific recombination by crossover between two distant Cre recognition sequences, i.e., loxP sites. A DNA sequence introduced between the two loxP sequences (termed floxed DNA) is excised by Cre-mediated recombination. Control of Cre expression in a transgenic animal, using either spatial control (with a tissue- or cell-specific promoter) or temporal control (with an inducible system), results in control of DNA excision between the two loxP sites. One application is for conditional gene inactivation (conditional knockout). Another approach is for protein over-expression, wherein a floxed stop codon is inserted between the promoter sequence and the DNA of interest. Genetically modified animals do not express the transgene until Cre is expressed, leading to excision of the floxed stop codon. This system has been applied to tissue-specific oncogenesis and controlled antigene receptor expression in B lymphocytes. Inducible Cre recombinases have also been developed. The inducible Cre recombinase is activated only by administration of an exogenous ligand. The inducible Cre recombinases are fusion proteins containing the original Cre recombinase and a specific ligand-binding domain. The functional activity of the Cre recombinase is dependent on an external ligand that is able to bind to this specific domain in the fusion protein.

Embodiments include an in vitro cell, an in vivo cell, and a genetically modified animal such as a livestock animal that comprise a gene under control of an inducible system. The genetic modification of an animal may be genomic or mosaic. The inducible system may be, for instance, selected from the group consisting of Tet-On, Tet-Off, Cre-lox, and Hif1alpha. An embodiment is a gene set forth herein.

### Dominant Negatives

Genes may thus be disrupted not only by removal or RNAi suppression but also by creation/expression of a dominant negative variant of a protein which has inhibitory effects on the normal function of that gene product. The expression of a dominant negative (DN) gene can result in an altered phenotype, exerted by a) a titration effect; the DN PASSIVELY competes with an endogenous gene product for either a cooperative factor or the normal target of the endogenous gene without elaborating the same activity, b) a poison pill (or monkey wrench) effect wherein the dominant negative gene product ACTIVELY interferes with a process required for normal gene function, c) a feedback effect, wherein the DN ACTIVELY stimulates a negative regulator of the gene function.

### Founder Animals, Animal Lines, Traits, and Reproduction

Founder animals (F0 generation) may be produced by cloning and other methods described herein. The founders can be homozygous for a genetic modification, as in the case where a zygote or a primary cell undergoes a homozygous modification. Similarly, founders can also be made that are heterozygous. The founders may be genomically modified, meaning that the cells in their genome have undergone modification. Founders can be mosaic for a modification, as may happen when vectors are introduced into one of a plurality of cells in an embryo, typically at a blastocyst stage. Progeny of mosaic animals may be tested to identify progeny that are genomically modified. An animal line is established when a pool of animals has been created that can be reproduced sexually or by assisted reproductive techniques, with heterogeneous or homozygous progeny consistently expressing the modification.

In livestock, many alleles are known to be linked to various traits such as production traits, type traits, workability traits, and other functional traits. Artisans are accustomed to monitoring and quantifying these traits, e.g., Visscher et al., Livestock Production Science, 40:123-137, 1994; U.S. Pat. No. 7,709,206; U.S. 2001/0016315; U.S. 2011/0023140; and U.S. 2005/0153317. An animal line may include a trait chosen from a trait in the group consisting of a production trait, a type trait, a workability trait, a fertility trait, a mothering trait, and a disease resistance trait. Further traits include expression of a recombinant gene product.

### Recombinases

Embodiments of the invention include administration of a targeted nuclease system with a recombinase (e.g., a RecA protein, a Rad51) or other DNA-binding protein associated with DNA recombination. A recombinase forms a filament with a nucleic acid fragment and, in effect, searches cellular DNA to find a DNA sequence substantially homologous to the sequence. For instance a recombinase may be combined with a nucleic acid sequence that serves as a template for HDR. The recombinase is then combined with the HDR template to form a filament and placed into the cell. The recombinase and/or HDR template that combines with the recombinase may be placed in the cell or embryo as a protein, an mRNA, or with a vector that encodes the recombinase. The disclosure of U.S. 2011/0059160 (U.S. patent application Ser. No. 12/869,232) is hereby incorporated herein by reference for all purposes; in case of conflict, the specification is controlling. The term recombinase refers to a genetic recombination enzyme that enzymatically catalyzes, in a cell, the joining of relatively short pieces of DNA between two relatively longer DNA strands. Recombinases include Cre recombinase, Hin recombinase, RecA, RAD51, Cre, and FLP. Cre recombinase is a Type I topoisomerase from P1 bacteriophage that catalyzes site-specific recombination of DNA between loxP sites. Hin recombinase is a 21 kD protein composed of 198 amino acids that is found in the bacteria Salmonella. Hin belongs to the serine recombinase family of DNA invertases in which it relies on the active site serine to initiate DNA cleavage and recombination. RAD51 is a human gene. The protein encoded by this gene is a member of the RAD51 protein family which assists in repair of DNA double strand breaks. RAD51 family members are homologous to the bacterial RecA and yeast Rad51. Cre recombinase is an enzyme that is used in experiments to delete specific sequences that are flanked by loxP sites. FLP refers to Flippase recombination enzyme (FLP or Flp) derived from the 2μ plasmid of the baker's yeast Saccharomyces cerevisiae.

Herein, “RecA” or “RecA protein” refers to a family of RecA-like recombination proteins having essentially all or most of the same functions, particularly: (i) the ability to position properly oligonucleotides or polynucleotides on their homologous targets for subsequent extension by DNA polymerases; (ii) the ability topologically to prepare duplex nucleic acid for DNA synthesis; and, (iii) the ability of RecA/oligonucleotide or RecA/polynucleotide complexes efficiently to find and bind to complementary sequences. The best characterized RecA protein is from E. coli; in addition to the original allelic form of the protein a number of mutant RecA-like proteins have been identified, for example, RecA803. Further, many organisms have RecA-like strand-transfer proteins including, for example, yeast, Drosophila, mammals including humans, and plants. These proteins include, for example, Rec1, Rec2, Rad51, Rad51B, Rad51C, Rad51D, Rad51E, XRCC2 and DMC1. An embodiment of the recombination protein is the RecA protein of E. coli. Alternatively, the RecA protein can be the mutant RecA-803 protein of E. coli, a RecA protein from another bacterial source or a homologous recombination protein from another organism.

### Compositions and Kits

The present invention also provides compositions and kits containing, for example, nucleic acid molecules encoding site-specific endonucleases, CRISPR, Cas9, ZNFs, TALENs, RecA-gal4 fusions, polypeptides of the same, compositions containing such nucleic acid molecules or polypeptides, or engineered cell lines. An HDR may also be provided that is effective for introgression of an indicated allele. Such items can be used, for example, as research tools, or therapeutically.

A majority of disease-causing or modifying HF genes initially identified encode proteins directly involved in cardiomyocyte contraction and cytoskeletal structure, (see, Table 1). An expanded understanding of disease pathobiology has emerged from genomic strategies, which have linked HF to impaired ion homeostasis and gene regulation [15]. Mutations in these genes, were initially discovered in individuals with familial HF including familial dilated cardiomyopathy and familial hypertrophic cardiomyopathy. For example, it is estimated that 750,000 people in the U.S. alone have DCM roughly half of which cases are familial. Of these familial cases it is estimated that more than 30 genes are involved with mutations in TTN accounting for approximately 20 percent of the cases. Similarly, familial hypertrophic cardiomyopathy is estimated to affect 640,000 people in the U.S. most importantly MYH7, MYBPC3, TNNT2 and TNNI3. Unfortunately, methods to identify and treat the disease are hard to develop as many of those having CM are not diagnosed until the disease is well advanced or fatal. Thus, the large animal genetic model system provided herein is an ideal model to create a clinically relevant and rapidly scalable large animal HF platform for identification and treatment of HF.

The inventors have developed an innovative gene editing platform for large animals that has achieved several significant accomplishments including: 1) seamless introduction of the orthologous human HF mutations (RBM20, R636S; BAGS, E455K; and TTN, domain deletions; for example) into the swine genome, 2) validation of homozygous, compound heterozygous and heterozygous mutant animals with significant clinical HF, 3) decreased survival in a dose response genotype/phenotype in F1 offspring, and 4) establishment of homozygotes, compound heterozygotes and heterozygotes for colony propagation. The pigs disclosed herein are the first genetic, large animal models of HF. These models enable the optimization of novel interventional strategies for HF including pharmaceutical, biologics and device strategies.

With RBM20, the scalable genetic model system has demonstrated early onset biventricular HF with correlation of phenotype and mutant allele burden. This first-of-a-kind model system provides a relevant, in-demand, reproducible tool to accelerate pharmaceutical studies focused on novel HF therapies and establish a pipeline for additional large animal models of human disease. At the molecular level, RBM20 has been linked to post-transcriptional regulation and the alternative splicing of TTN and CAMK2D in cardiac tissue [4, 17, 18]. An essential role of RNA binding proteins in cardiac function and developmental processes has been established in other studies. For example, RBM24 was recently demonstrated to be enriched in embryonic stem cell (ESC)-derived cardiomyocytes and required for sarcomere assembly and heart contractility [19]. In addition, Hermes was found to regulate heart development in Xenopus [16]. Furthermore, LIN28, DAZL, and GRSF1 contribute to maintenance of pluripotency and differentiation of ESCs [20, 21] while coordinated teamwork of CELF and MBNL1 proteins are critical during normal cardiac and skeletal muscle development [22]. Based on these findings, RNA processing is established as a major regulator of early gene expression machinery during developmental processes and may underlie the progenitor cell contribution to ongoing cardiac homeostasis and tissue renewal [23]. These fundamental components are dependent on proper RBM20 and alterations in RBM20 provoke molecular disruption during cardiogenesis and continue to dysregulate cardiac protein homeostasis in mature cardiac tissues.

RBM20 is an essential component of the RNA processing machinery during cardiogenesis and is required to regulate cardiac gene expression to pattern normal structural and physiological integrity of newly formed cardiomyocytes [3]. Utilizing this cardiogenic pipeline based on patient-specific pluripotent stem cells, the inventors have established an in vitro model of RBM20-linked DCM and unmasked the initial molecular and cellular dysfunctions [8]. This approach established RBM20 disruption as an early onset mechanism of human cardiomyopathy with altered early stage cardiogenic gene expression, calcium overload, sarcomeric abnormalities, and gene expression patterns of compensated HF. These defining features of RBM20-deficient cardiomyopathy recapitulate important universal features of HF due to a cardiomyocyte-based genetic disease that can be further examined in a large animal system for targeted approaches applicable to a wide spectrum of industries focused on cardiovascular medicine.

Heart failure model systems traditionally employ labor-intensive strategies with technical variations, i.e. surgical constrictions such as pulmonary artery or aortic banding, as well as pacing induced tachycardia. Therefore, an HF model system that is scalable, predictable, and reproducible better enables clinical translation of novel devices and pharmaceutical innovation and addresses the high-costs and low-throughput of inducible HF models. Furthermore, induced model systems cannot fully recapitulate the chronic nature of cardiomyopathy that invariably affects biventricular heart disease. Genetically engineered cardiomyopathy offers a transformational approach to address these limitations.

Ventricular tachycardia (V-tach or VT) is a type of regular and fast heart rate that arises from improper electrical activity in the ventricles of the heart. [“Types of Arrhythmia”, NHLBI. Jul. 1, 2011. Archived from the original on 7 Jun. 2015.] Although a few seconds may not result in problems, longer periods are dangerous. [Id.] Short periods may occur without symptoms or present with lightheadedness, palpitations, or chest pain. [Baldzizhar, A; et al. (September 2016). “Ventricular Tachycardias: Characteristics and Management.”. Critical care nursing clinics of North America. 28 (3): 317-29.] Ventricular tachycardia may result in cardiac arrest and turn into ventricular fibrillation. [“Types of Arrhythmia”][Baldzizhar et al.] Ventricular tachycardia is found initially in about 7% of people in cardiac arrest. [Baldzizhar et al.]

Ventricular tachycardia can occur due to coronary heart disease, aortic stenosis, cardiomyopathy, electrolyte problems, or a heart attack. [“Types of Arrhythmia”][Baldzizhar et al.] Diagnosis is by an electrocardiogram (ECG) showing a rate of greater than 120 bpm and at least three wide QRS complexes in a row. It is classified as non-sustained versus sustained based on whether or not it lasts less than or more than 30 seconds. The term “ventricular tachycardias” refers to the group of irregular heartbeats that includes ventricular tachycardia, ventricular fibrillation, and torsades de pointes. [Id.]

In those who have a normal blood pressure and strong pulse, the antiarrhythmic medication procainamide may be used. [Id.] Otherwise immediate cardioversion is recommended. [Id.] In those in cardiac arrest due to ventricular tachycardia cardiopulmonary resuscitation (CPR) and defibrillation is recommended. Biphasic defibrillation may be better than monophasic. While waiting for a defibrillator, a precordial thump may be attempted in those on a heart monitor who are seen to go into an unstable ventricular tachycardia. [Neumar, R W; et al. (3 Nov. 2015). “Part 1: Executive Summary: 2015 American Heart Association Guidelines Update for Cardiopulmonary Resuscitation and Emergency Cardiovascular Care.” Circulation. 132 (18 Suppl 2): S315-67.] In those with cardiac arrest due to ventricular tachycardia survival is about 45%. An implantable cardiac defibrillator or medications such as calcium channel blockers or amiodarone may be used to prevent recurrence. [Baldzizhar et al.]

### Custom Swine Models for Cardiovascular Innovation.

Continued innovation in human cell based regenerative medicine has led to a dramatic increase in the number of new cell-based investigational drugs (INDs) submitted to the FDA. Between 2006 and 2013, 163 INDs involving cell-based therapies were filed with a range of clinical indications, the largest proportions of which related to cardiovascular therapy (27% of INDs) [24]. The number of new submissions is rising and is expected to continue into the foreseeable future. These cell-based therapies are very heterogeneous with differences in the source of the therapeutic cells, isolation and treatments of the cells, dosage and delivery of the cells. Therefore, cellular therapy has to pass through several levels of preclinical testing to justify human clinical trials. Preclinical evaluations should ideally 1) establish the scientific rationale for the therapeutics, 2) investigate the route of administration and characterize local and systemic toxicities of the therapeutic agent, 3) carry out dosage escalation studies to determine the dosing range and a safe starting dose for clinical trials and 4) determine which groups of patients to the therapeutic regimen could benefit and establish a clinical monitoring scheme. Choosing the correct animal model for preclinical testing is critical to generate the most relevant results. Whenever feasible, the proposed therapeutics should be tested in a model with the greatest similarity in disease state, anatomy and physiology as the target patient. For this reason, swine are the most commonly used large animal model for preclinical cardiovascular evaluation of novel therapeutics ranging from drugs and cell-based regeneration to mechanical assist and resynchronization devices.

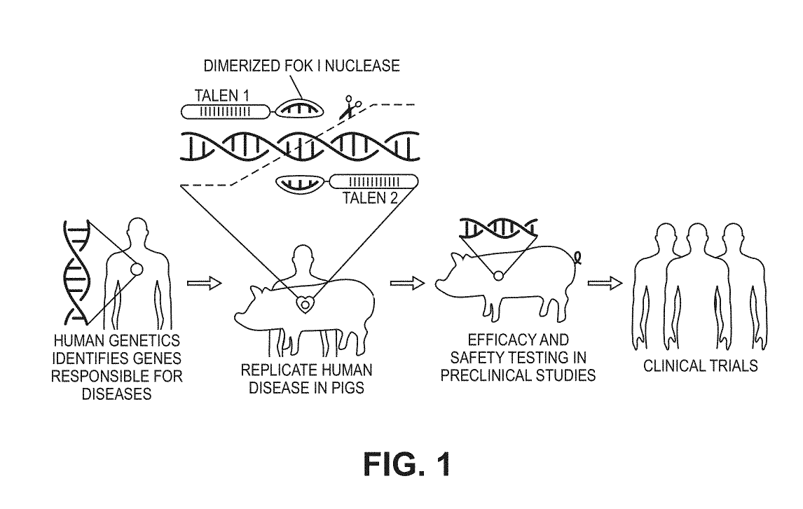

However, unlike rodents, no catalogue of swine with the desired disease state can be readily accessed for testing of novel therapeutics. The present invention changes this by precisely editing the swine genome to mimic a variety of human cardiovascular disease states, particularly those with the highest demand based on the ability to closely recapitulate the genotype/phenotype relationships established in clinical cohorts including one or more of those genes identified in Table 1. The disclosed methods utilize a suite of highly effective genome engineering methods to develop swine models that precisely reproduce human disease conditions and greatly increase the utility of swine in preclinical testing (FIG. 1). This paradigm was utilized to develop the initial RBM20 mutant swine as a model of HF. This well-defined disease-causing genetic mutation in patients with inheritable cardiomyopathy is characterized as highly penetrant, early-onset, and severe myopathic features common to other DCM patient cohorts with a common final pathway of HF. Therefore, this genetic cause of cardiomyopathy will provide a platform for novel therapeutic testing for pediatric HF as well as adult HF. This validated large animal model system for cardiomyopathy will establish a first-in-class tool for IND-enabling studies addressing Pharmacology/Toxicology studies mandated by the FDA.

Ossabaw miniature swine enable HF modeling with comorbid Metabolic Syndrome. Metabolic syndrome (MetS) or “pre-diabetes” currently afflicts up to 27% of the United States population, is drastically increasing in prevalence here and abroad [25] and often progresses to Type-II diabetes. In Asian populations there is a much higher incidence of HF in DM Type II patients (40%) than in western populations (20%). Furthermore, in Singaporean and Indian populations, there is an 80% incidence of HF in DM Type II patients. Therefore, there is an urgent need to develop large animal models with comorbid MetS. Yucatan swine are the most commonly used miniature swine for study of cardiovascular disease due to their breed familiarity. However, this commonly used model does not develop metabolic syndrome through dietary manipulation and does not progress to Type-II diabetes [26-29]. In contrast to the Yucatan pig model, the recently re-discovered Ossabaw minipig shows all six hallmarks of metabolic syndrome, including; central obesity, insulin resistance, impaired glucose tolerance, dyslipidemia, and hypertension [30]. The inventors propose that for these reasons that the Ossabaw minipig is an ideal, small stature breed for production of HF pigs. As discussed above, this is particular benefit in those cases of CM where the onset, severity or treatment of the disease is complicated by metabolic dysfuncton arising from hypertension, diabetes and other forms of heart disease.

While Ossabaw swine provide many advantages, large white swine also provide multiple advantages (i) conventional swine have been used in current research programs, thus control datasets are readily available and (ii) conventional swine have exceptional fecundity compared to miniature swine with an average litter size near 10 versus 4. Furthermore, the conventional swine with early onset genetic cardiomyopathy enables optimal modeling for childhood HF with rapid cardiac growth modeling the physiological demands of adolescent growth in humans within the first three months of life for the swine. However, a limitation of conventional swine is size, with adults of 1+ years exceeding 200 kg. For studies aiming to treat early onset HF and/or shorter-term studies, the size of conventional swine background will be ideal to reduce costs and time of a large animal study design. In the case of long-term studies, a breed with small stature will be desirable; hence, another rationale for developing RBM20 mutant Ossabaw swine. Finally, the penetrance and severity of HF mutations is expected to vary with background genetics. Creation of the model in two lines could produce animals with unique pathologies that will better fit the intended study design for a wide spectrum of academic and commercial customers.

### Example 1: Identify RBM20 Mutational Hotspot in Swine

The inventors have developed RBM20-R636S homozygotes and heterozygotes swine that recapitulate dose-dependent genotype/phenotype clinical endpoints. As predicted, DCM is more severe in the homozygotes with a high rate of neonatal mortality or early onset of cardiomyopathy mimicking pediatric disease [2]. The inventors have played a key role in defining the molecular etiology of DCM in RBM20 deficient model systems [3, 8], and are uniquely positioned with preclinical assessment of porcine-induced cardiomyopathies [9]. Using these methods and tools, the inventors can accelerate the discovery and translation of novel therapeutics for HF by establishing a reproducible large animal genetic model with a clinically relevant phenotype by following the Examples as discussed below.

### Characterization of Heart Failure Progression in RBM20-R636S Gene Edited Conventional Swine.

12-14 offspring from conventional R636S mutant swine are evaluated with wild type, homozygous, and heterozygous genotypes for up to 24 weeks. Functional analysis includes cardiac electrophysiology, echocardiography, and cardiac MRI. Functional physiological studies will be corroborated by pathological analysis at study summation. The molecular profile of disease progression is precisely examined through RNA-seq from both homozygotes and heterozygotes with predictable HF onset. Accurate characterization of the molecular pathogenesis provides additional therapeutic insights and aid in development of biomarkers to monitor disease progression.

The first step towards building a clinically relevant HF model in engineered pigs was to (i) identify the mutational “hotspot” in the swine gene and (ii) determine if key residues implicated in DCM were conserved. The majority of pathological mutations of RBM20 are localized to the RS-rich domain, consisting of 5 amino acids, RSRSP. Alignment of swine and human RBM20 revealed 83% homology over the entire protein, perfect conservation of the RS-rich domain and the 45 amino acids flanking the RS-rich domain (not shown). Considering this conservation and the strong data supporting a conserved role of RBM20 in mouse and rodent knock-outs, the inventors hypothesized that point mutations in the RS-rich domain would have the same pathological effects in swine as previously characterized in clinical cohorts of DCM.

### Example 2: Mimic the Human R636S Mutation in Porcine Cells by Gene Editing

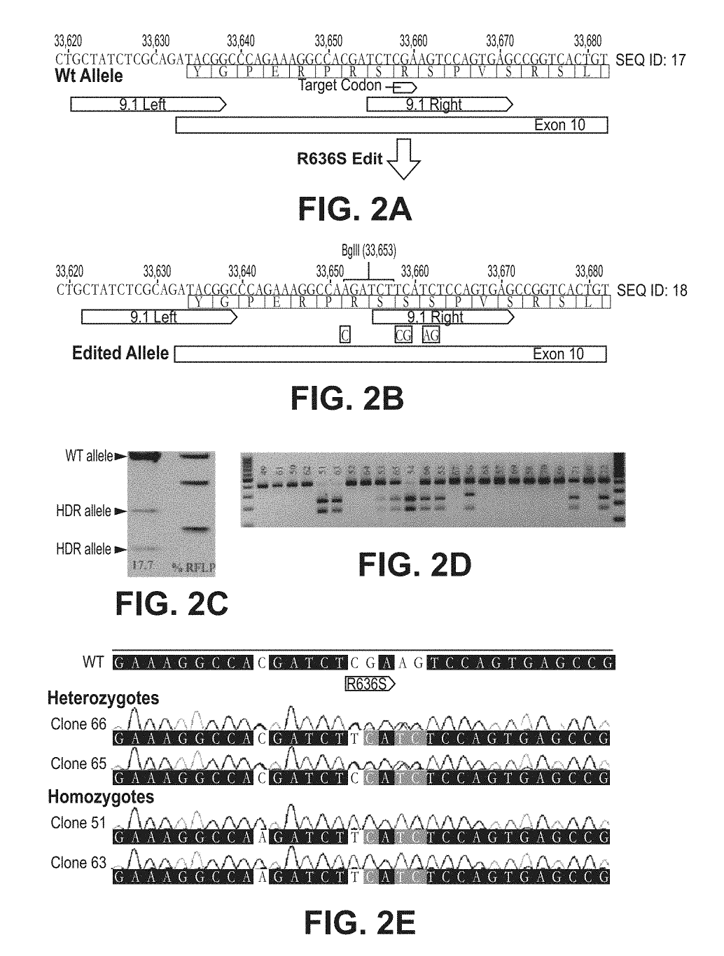

The inventors chose to mimic the R636S mutation in swine due to the severity and predictability of clinical cohorts. TALENs were designed to the wild type sequence such that the binding site of the right monomer based on an innovative design strategy for introgression SNP edits (FIG. 2A-2E). The repair template directs nucleotide changes to code for the R636S, and also silent mutations to create a novel BglII restriction site for restriction fragment length polymorphism (RFLP) genotyping. The resulting edited allele (FIG. 2A-2E) has four novel SNPs; hence, will be no longer be a suitable substrate for TALEN cleavage. This design is the product of testing several iterations of repair template/TALEN combinations to maximize efficient HDR without introducing confounding indels on the edited allele, a common occurrence in TALEN or CRISPR HDR [31].

Whereas use of TALEN or CRISPR nickases would theoretically prevent this problem, the inventors have demonstrated nicks to be a poor substrate for HDR in pig fibroblast limiting their utility (data not shown). Using an optimized design, the inventors were able to achieve 18-30% HDR by RFLP in different populations of cells. Individual colonies were isolated from the population with 30/144 (21%) and 5/144 (3.4%) genotyped as heterozygous (HTZ) or homozygous (HMZ) by the RFLP assay. From this group, 17 and 3 of the candidates respectively were sequence validated and able to be cryopreserved for cloning (Table 2). The process was repeated in male landrace cells as well as male Ossabaw minipig cells and the resulting lines are available for cloning (Table 3) the TALENs sequences, binding domains and HDR template sequences are provided in Tables 3 and 4 respectively.

TABLE 2
Established RBM20 mutant clones.

| Trial | Mutation | Sex, Breed | HTZ | HMZ |
|---|---|---|---|---|
| Rbm1 | R636H | Male, Ossabaw | 4 | 2 |
| Rbm4 | R636S | Male, Ossabaw | 2 | 0 |
| Rbm9 | R636S | Female, Landrace | 17* | 3 |
| Rbm10 | R636S | Male, Landrace | 9 | 0 |
| Rbm11 | R636S | Male, Landrace | 40* | 9* |

TABLE 3

| TALEN | | Nucleotide |
|---|---|---|
| Name | RVD Sequence | Sequence (5′-3′) |
| ssRBM20 | NN HD NG NI NG | GCTATCTCGCAGATAC |
| 9.1 L | HD NG HD NN HD | GG (SEQ ID: 1) |
| | NI NN NI NG NI | |
| | HD NN NN | |
| ssRBM20 | HD NI HD NG NN | CACTGGACTTCGAGA |
| 9.1 R | NN NI HD NG NG | (SEQ ID: 2) |
| | HD NN NI NN NI | |

Table 4 provides sequences of HDR templates used to make the identified mutations.

TABLE 4

| Oligo Name | Nucleotide Sequence (5′-3′) |
|---|---|
| ssRbm20 R to H | agctgctctgctatctcgcagatacggcccag |
| [RBM1] | aaaggccaAgatctcACagtccagtgagccgg |
| | tcactgtccccgaggtcccacactcc |
| | (SEQ ID: 3) |
| ssRbm20 R to S | agctgctctgctatctcgcagatacggcccag |
| [RBM4] | aaaggccaAgatctTCaagtccagtgagccgg |
| | tcactgtccccgaggtcccacactcc |
| | (SEQ ID: 4) |
| ssRBM20 R to S | agctgctctgctatctcgcagatacggcccag |
| (2MM) [RBM9, | aaaggccaAgatctTCaTCtccagtgagccgg |
| RBM10, RBM11] | tcactgtccccgaggtcccacactcc |
| | (SEQ ID: 5) |

### Example 3: Establish RBM20 Mutant Pigs by Cloning

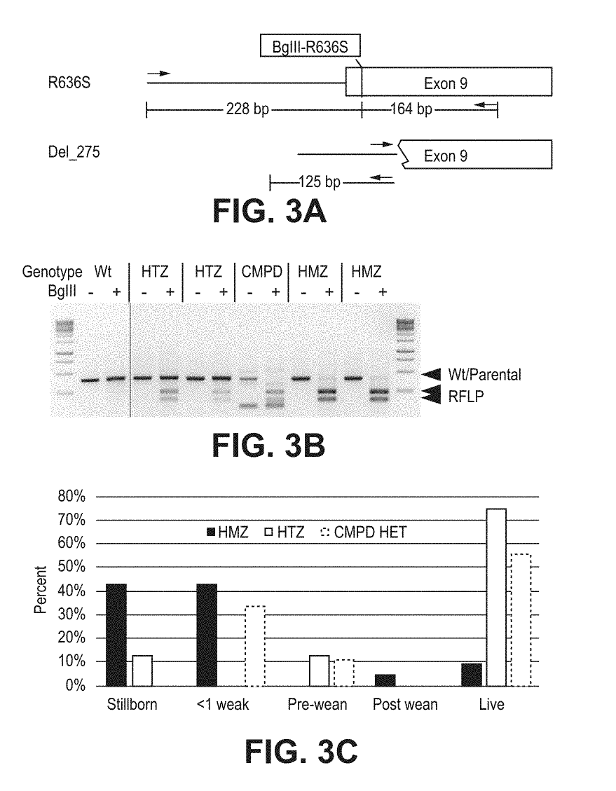

It is recognized that the success rate of cloning was stochastic and could be further confounded by unknown severity of the HF phenotype. This possibility was addressed as follows: 1) specifically chose to create pools of both HTZ and HMZ clones where it is expected that the latency of disease to be much greater in the HTZ animals and, 2) several positive colonies from a successfully cloned donor protects from the significant variation in the success of somatic cell transfer from individual clones [32]. The inventors have successfully utilized this approach for development of LDLR, APC and DAZL gene-edited pigs [31, 33]. Pools of RBM9 and RBM11 (Table 2—denoted with asterisk) were cloned and the resulting embryos were transferred to four synchronized recipients each. Ten of the twelve recipients were pregnant at day 30 of gestation, and eight pregnancies went full term. From the HMZ pools, a total of 29 piglets were farrowed, and from the HTZ pool, eight piglets farrowed from the single, full term pregnancy. Each piglet was evaluated by RFLP assay and sequencing to confirm the edited genotype. Most animals had the expected R636S HTZ or HMZ genotype (FIG. 3A-3C). Another group of animals from the same cells were found to be compound heterozygotes (CMPD) consisting of one allele R636S allele and a second knockout allele resulting from a 275 bp mutation (Del_275). These cells were pooled with the RBM11 cells in Table 2 for cloning.

### Example 4: Establish RBM20 Mutant Initial Breedstock

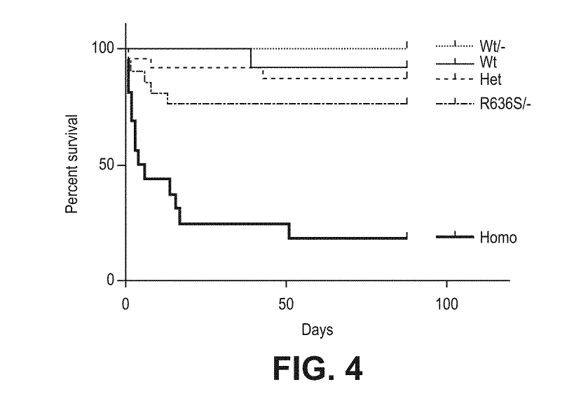

From this breedstock, production was expanded of three distinct genotypes (FIG. 3A-3C) that have different pathological features or disease latency of value to preclinical studies. Accordingly the invention, also provides HMZ and HTZ and CMPD hets that have now been produced from a traditional breeding program and are now available for all subsequent experimentation for early onset HF and latent HF of these three genotypes. FIG. 4 is a Kaplan-Meier plot estimating the survival function of various genetically modified animals according to the invention. As shown, the survival of the F1 generation demonstrates a gene dose dependent phenotype (FIG. 4) for RBM20 heterozygous, homozygous for the R636S mutation, and compound heterozygotes (CMPD) with one KO allele and one R636S mutant allele. Wild-type swine have essentially 100% survival in the first 12 weeks of life without traumatic injury during the neonatal period. However, there is a strong dose dependent genotype/phenotype correlation with RBM20 mutations. Homozygous animals (solid black) have a ˜25% survival at 12-weeks with the majority of mortality occurring with sudden neonatal death. Heterozygous animals (large/small dash line) have ˜80% survival at 12-weeks. Survival of compound heterozygotes is intermediate between homozygotes and heterozygotes. The ability to create animals of varying genomic modifications is a major benefit to the instant invention as it provides the ability to test a therapeutic on different genotypes of a disease or different severities of a disease, where for example, a heterozygote may present a disease arising from a particular mutation less severely or with different pathologies than a homozygote or a compound heterozygote thus allowing a dissection of the causative effects of any particular allelic mutation to a pathology as a whole. In addition, less severe phenotypes may better enable longitudinal study of therapeutic interventions.

### Example 5: Define Pathological Features of RBM20 Mutants

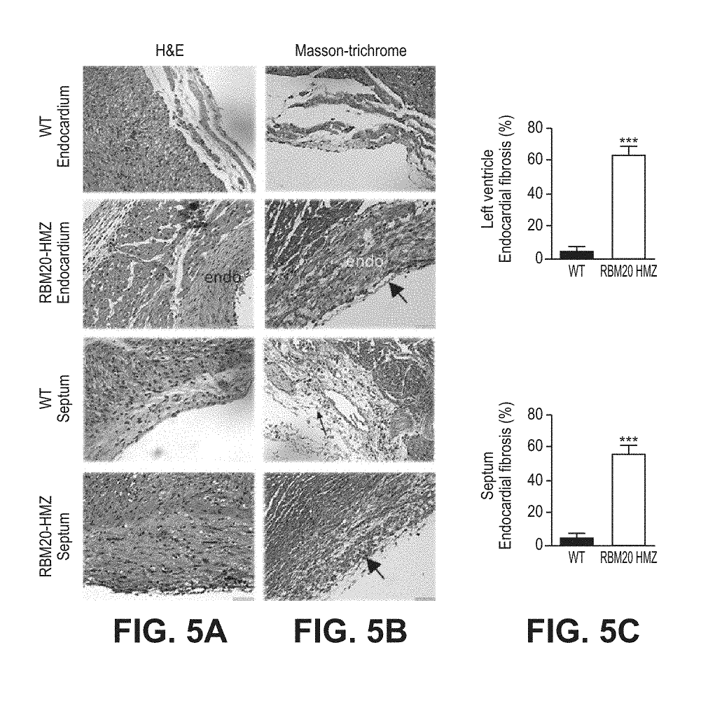

Gross pathology was observed upon immediate necropsy of stillborn fetuses or piglets that died within the first week of life. Based on board certified veterinarian assessment, three notable cardiovascular features were reproducibly observed in HMZ without any gross abnormalities in other organ structures. 1) Hemopericardium was universal upon opening the chest cavity without evidence of dissection in great vessels suggesting a possible coronary or small vessel disease process, 2) cardiac enlargement with patchy areas of opacity was grossly notable that was consistent with infant DCM without evidence of hypertrophy, and 3) endocardium was pale, white fibrotic appearing. A cardiac pathologist reviewed histology slides in a blinded fashion. Through histological analysis, a severe subendocardial fibrosis in HMZ animals has been demonstrated (FIG. 5A-5C).

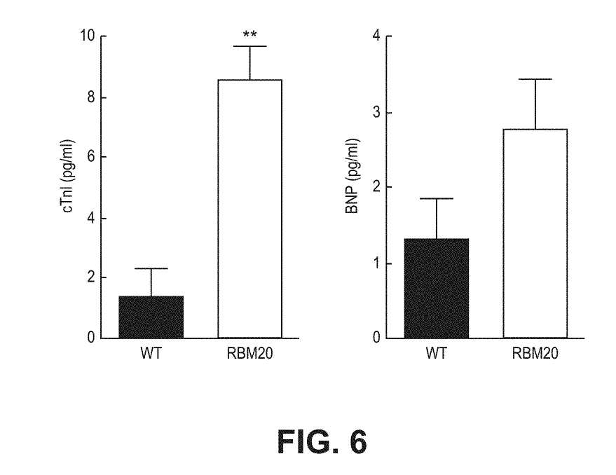

Despite the high mortality rate of R636S homozygotes and compelling histopathology, two animals have thus-far survived with this genotype. Notably, serum cardiac troponin I (cTnI) and brain natriuretic peptide (BNP) levels, biomarkers of myocardial damage and heart failure, are elevated in mutant animals (FIG. 6). Though somewhat surprising that homozygotes are able to survive, intrafamilial variability in penetrance among patients with the same mutation to the RS-rich domain has been observed and a subset have not yet developed DCM, even as young adults (2). In addition, RBM20 deficient rodents do not manifest significant disease until 1+ years of age (17, 34). Epigenetic reprogramming in cloned pigs is stochastic process and often incomplete (37, 38) leading to altered gene expression (39), aberrant penetrance of x-linked SCID (40). Thus, it will be interesting to evaluate whether epigenetic modifications also play a role in RBM20 mutant phenotypes, a variable that is amplified by cloning. In addition, decreased cardiac output and/or congestive heart failure have been observed in some cloned swine, which could have influenced the myocardial pathology in neonatal pigs (35, 36). We hypothesize that the surviving homozygous clones serendipitously escaped the severe, early onset DCM via altered gene expression and compensations related to aberrant epigenetic reprogramming. These animals may be of critical importance as uncovering the mechanism of escape could predict novel druggable targets to prevent splicing related CM. Therefore, these phenotypic outliers will be maintained and closely monitored for future mechanistic studies with academic partners with expertise in epigenetics with the hope that additional commercial applications can be further developed, yet this is outside of the scope for this application.

Despite the high mortality rate of cloned R636S HMZ and compelling histopathology, numerous HTZ remain from the initial cloning process. The inventors now have F1 progeny from HTZ breeding pairs that have produced over 20 offspring. The survival rate for the F1 RBM20 HMZ is significantly reduced within the first 3 months of life compared to a zero mortality rate of wild-type (WT) and a 20% mortality rate for F1 HTZ RBM20 (FIG. 3A-3C). This is in contrast to RBM20 deficient rodents that did not manifest significant disease until 1+ years of age [17, 34], but similar to human patient cohorts with early onset HF in infancy or childhood. Epigenetic reprogramming in cloned pigs is a stochastic process and often incomplete [35, 36] leading to altered gene expression [37] and aberrant penetrance of x-linked SCID [38]. Thus, it will be important to evaluate whether epigenetic modifications also play a role in RBM20 mutant phenotypes, a variable that is amplified by cloning. The inventors submit that the F1 offspring from the original cloned animals will continue to provide a reliable and reproducible disease phenotype compared to the stochastic behavior of the original cloned animals. Including, the addition of naturally breed offspring provides greater confidence in the ability to provide naturally born HTZ, CMPD and HMZ animals.

### Example 6: Establish Stress-Response in RBM20-R636S Gene Edited Conventional Swine

The inventors will evaluate 10-12 offspring of both homozygotes and heterozygotes compared to wild type offspring following acute physiological stress at 8 and 24 weeks of age to determine cardiac performance and contractility of the diseased myocardium. Clinical-grade event recorders and telemetry is used to capture the cause of sudden death and quantitatively document the burden of arrhythmias before and during imposed stress tests.

The HMZ swine model with an early onset cardiomyopathy within 8 weeks of age is uniquely suited for studies of childhood DCM as the disease is manifested early, and the dramatic growth in the first three months emulates a decade of childhood growth in humans. This allows technologies to be evaluated as the heart grows, yet in a short time span. The breeding of HMZ swine of this model will now provide sufficient numbers of animals to also provide acute stress response studies to more precisely map the deficiencies in myocardial contractility with invasive testing. The Ossabaw miniature swine model has added importance in its ability to enable adult-sized animals to be used for pharmaceutical and device testing and for studies in a MetS comorbid state.

### Characterization of HF Progression in R636S Gene Edited Conventional Swine

As discussed in Examples 2, 3 and 4, F1 HMZ, CMPD and HTZ are now available. The next steps are to characterize the reproducibility of the genotypes according to the age of HF onset and biventricular heart disease in at least 10-12 animals for each genotype of HMZ, CMPD, HTZ, and WT animals. This will be achieved with an increased production schedule of F1/F2 offspring from the breeding stock now available. It is then possible to functionally analyze cardiac performance over 6-months using non-invasive cardiac electrophysiology, echocardiography, and cardiac MRI modalities. These animals and phenotypes will provide the tissue samples for molecular profiling of disease progression and histological quantification.

### Example 7: Clone and Breed Conventional Swine with RBM20 R636S Mutations

Founder boars have been produced by cloning and have successfully produced F1 offspring. Semen from boars exhibiting normal fitness (no apparent fatigue associated with HF) will be collected for artificial insemination of wild type females. For the conventional lines, at least 10 HTZ females for the breeding herd should be a production target and expanding to 15+ in the second paradigm. Considering an average litter size of 10 and that 25% of the offspring will be female and HTZ for R636S, 4-6 wild type gilts will be bred. It is expected a breeding herd of 10 bred to HTZ males will produce about 60 HMZ and 120 HTZ models per year. It will also be possible for the inventors to use DAZL KO pigs [31] as recipients for germline stem-cell transplantation of HMZ R636S spermatogonial stem cells at 8-12 weeks of age. See U.S. Pub App 2014-0123330, the contents of which are hereby incorporated by reference in their entirety. The germ-cell free DAZL-KO boars are a resource unique to the applicant, Engrafted R636S cells will produce only R636S sperm allowing breeding as HMZ from a healthy surrogate boar (FIG. 8A-8F). This innovation has the benefit of reducing production cost as the same breeding herd of 10 could now produce 120 of both HMZ and HTZ animals per year. This will be sufficient for characterization as well as initial commercialization. The KO alleles will be maintained by minimal breeding and semen cryopreservation. A small cohort will be bred to homozygosity for molecular comparison to R636S mutants. The 50% mortality in the F1 generation due to genuine heart failure within 12 weeks of HMZ animals and a 20% mortality in HTZ at 12 weeks, is extremely encouraging, significant gene-dependent cardiovascular morbidity is evident, and yet it is still possible to scale the production with a traditional breeding program.

### Example 8: Monitor Cardiovascular Performance of RBM20 R636S Cohorts for 6-Months According to Standardized Operating Procedures

Baseline values of conventional swine have been previously established on over 60 animals using clinical chemistry, echocardiograms, rhythm monitoring, and cardiac MRI. Dramatically decreased survival rate has been noted with HMZ R636S in the RBM20 gene along with severe perinatal cardiac fibrosis. It is expected that the original F0 offspring from the cloning process will demonstrate the widest spectrum of variability as the F1 generation has now been produced and successfully analyzed.

All piglets are genotyped at birth and monitored at least twice daily to determine if any require euthanasia and tissue collection. Additionally, the animals will have cardiac imaging with both echocardiography and cardiac MRI to monitor cardiac function longitudinally. The inventors have produced and characterized their first litters of F1 with WT, HTZ, and HMZ RBM20 mutations out to 8 weeks of age. These data demonstrate the F1 progeny have a significantly more consistent and reproducible phenotype than the original cloned F0 progeny. This demonstrates the feasibility of the breeding program and significantly increases the reproducibility of the model system.

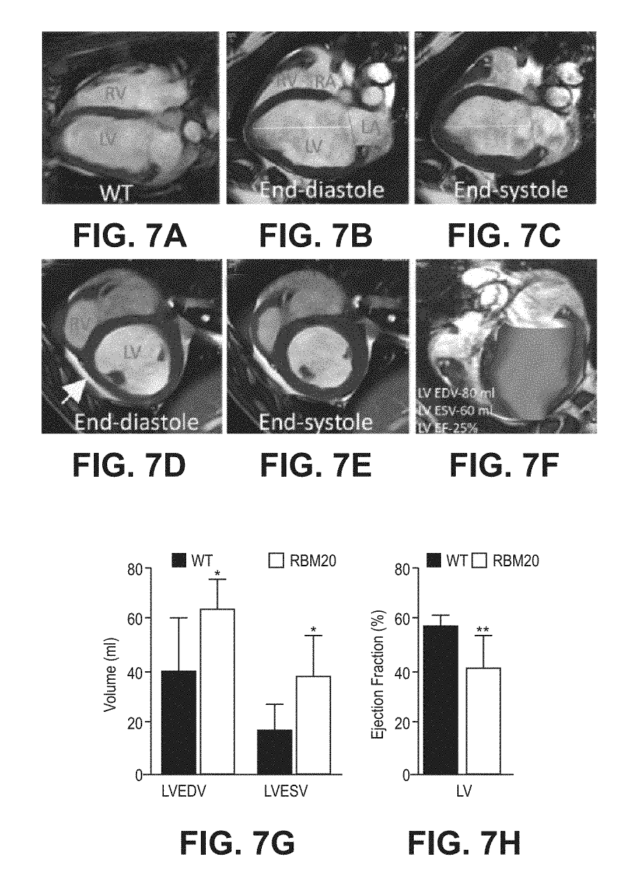

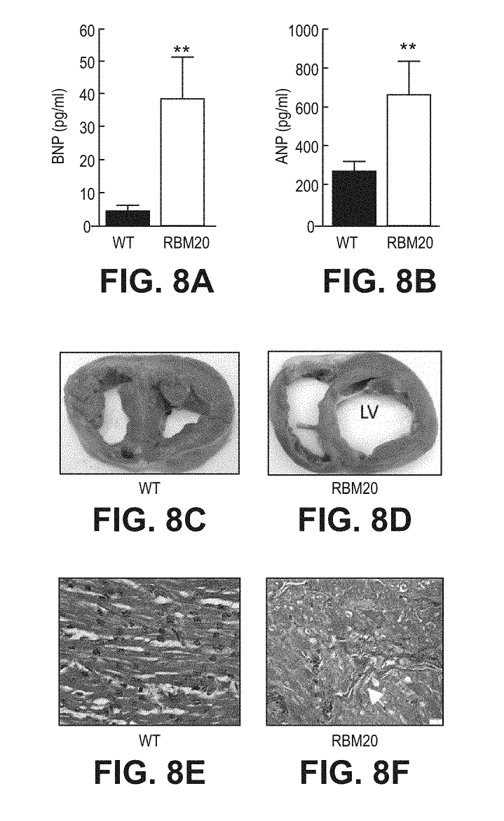

A significantly decreased survival rate of HMZ R636S F1 offspring compared to both WT and HTZ offspring has been demonstrated with this model, see, FIGS. 3A-3C and FIG. 4. This provides confidence in the reliability of this HF model system. Additionally, a double-blinded randomized follow-up analysis clearly delineated genotype/phenotype associations with significant heart failure in the HMZ animals within 8 weeks of life by functional MRI (FIG. 7A-7H) with HTZ demonstrating an increased heart rate compared to WT and a trend towards decreased LV and RV ejection fraction at 8 weeks (data not shown). This indicates that F1 HMZ mutants may provide an early onset disease phenotype that may mimic childhood heart failure and the HTZ mutants may provide a latent onset disease progression that could better model adult or acquired phenotypes. The ongoing analysis will determine the natural history of disease.

Additionally, these F1 offspring have demonstrated, by 8-weeks of age, a biventricular heart disease pattern that is genotype dependent. Using MRI, the right ventricle ejection fraction decreases from 61% to 52% and 49% with HTZ and HMZ genotypes, respectively. This indicates that as LV dysfunction progresses that there may be a measurable decrease in RV function in this genetic model system of HF. Biventricular heart failure is a critical importance in any model system of heart failure. The engineered model system here develops right and left heart dysfunction together.

It is anticipated that HTZ will progressively develop worsening heart disease within 6 months of careful follow-up and express a latent HF phenotype based on these initial studies. However, additional physiological stress may be used to express a measurable phenotype, providing a unique stress intolerant model system for therapeutics aiming to avoid disease progression and provide primary prevention.

### Example 9: Histopathology and Molecular Characterization of Cardiac Tissue to Determine Severity of Disease and Molecular Diagnostics

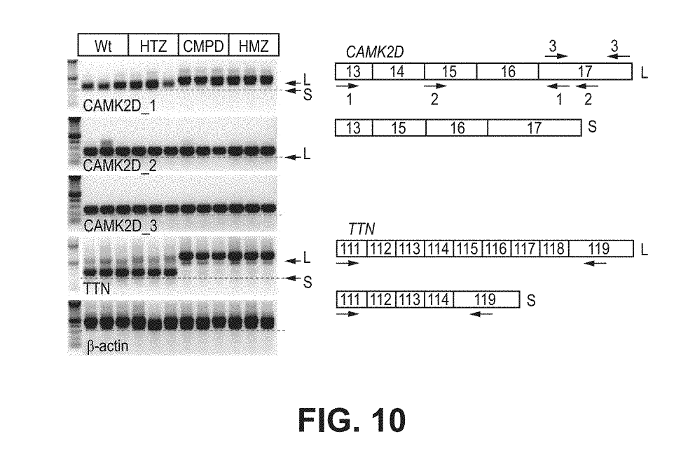

Establishing the molecular markers of disease progression is extremely helpful for future therapeutic testing protocols of this large animal model system of HF. There are two groups that will be most useful in developing companion diagnostics for this model system: 1) severe phenotype with early disease and demise compared to 2) phenotypically normal animals at 6-months despite confirmed genotype. Blood and cardiac tissue will be collected for mRNA extraction at the time of euthanasia or termination of the study. RNA will be processed for RNAseq to quantify expression levels that are significantly different as compared to WT samples as well as crosslinking immunoprecipitation (CLIP-seq). Serum samples will also provide a biorepository for electrospray mass spectroscopy for biomarker analysis, especially given preliminary data that demonstrates splicing defects in CAMK2D and TTN isoforms (FIG. 10) as previously reported (data not shown).

The inventors anticipate early onset fibrosis that becomes progressively deleterious to cardiac function (FIGS. 8A-8F). It is anticipated that increased fibrosis will correlate to increased arrhythmia documented with implantable loop recorders (ILRs). If cardiac fibrosis is severe enough in aging animals, the inventors will be able to detect and monitor by delayed enhancement on cardiac MRI. Because ILRs are not compatible with MRI, protocols will primarily assess cardiac function by echocardiography. However, if structural and functional abnormalities are noted, then ILRs will be surgically removed for cardiac MRI analysis. It will then be possible to correlate arrhythmias with delayed enhancement on MRI. This may provide a primary endpoint for therapeutic studies to reverse fibrosis and prevent electrical instability.

### Example 10: Report Genotype/Phenotype Associations

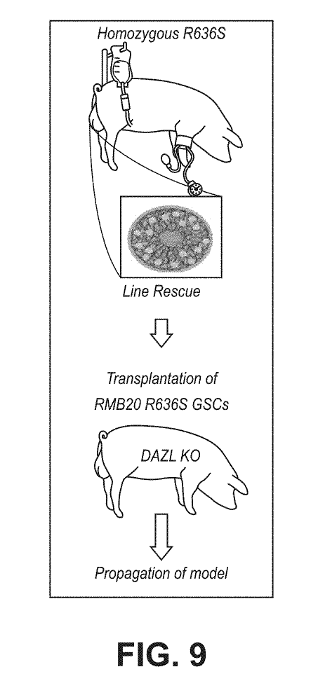

The genotype will be captured as soon as possible after birth; however, data will be stored in Metadata RAVE customized database by the research team blinded to genotype. This will ensure there is no selection bias in the data collection or interpretation of individual animals. Upon the death of an animal or 6-month conclusion of the study, data collected will be analyzed according to genotype cohorts. It is anticipated that refined piglet care will increase the survival rate of HMZ RBM20 animals in subsequent breeding yet preserve spontaneous development of the HF phenotype without the need for induced cardiac stress (FIG. 9).

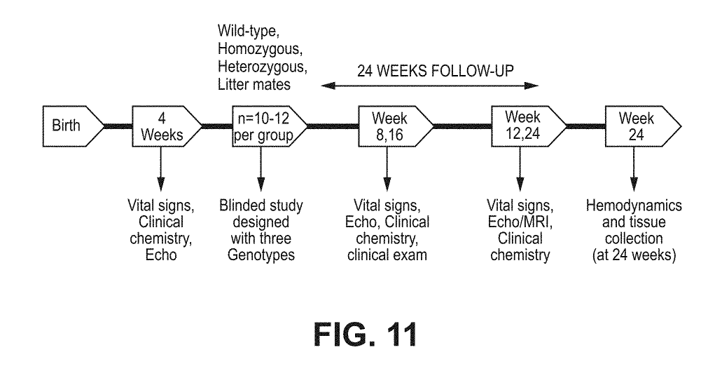

An unknown factor at this point is whether the health of RBM20 HTZ will impact the breeding success rate. At this point, the 21-month-old HTZ are in good health with no signs of decline and have produced one litter each. However, pregnancy and farrowing could add stress to the heart, and reduce the production lifespan of RBM20 sows. If this is the case, a more aggressive sow replacement scheme may be adopted instead of the typical 4-6 parodies and/or increase the size of the breeding herd. For males, the solution will be to transplant germline stem cells from HMZ RBM20 R636S boars into DAZL KO (FIG. 11).

### Example 11: Establishing a Stress-Response in RBM20-R636S Gene Edited Conventional Swine

Littermates are evaluated at 10 HMZ, 10 HTZ, and 10 WT for arrhythmias and physiological response to acute cardiovascular stress to determine cardiac performance and contractility of the diseased myocardium in adult swine at 24 weeks of age. Invasive hemodynamics will be collected in 10, HMZ, 10 HTZ, and 10 WT animals at 8 weeks of age to document the phenotype in juvenile animals in non-survival acute studies that can be age-matched with non-invasive studies. There is a high prevalence of sudden cardiac death in RBM20 mutation carriers presumably due to electrical disturbance. Clinical-grade ILRs and telemetry will be used to capture the cause of sudden death and quantitatively document the burden of arrhythmias. With 50% mortality of HMZ mutants, capturing 3-4 sudden deaths are anticipated with ILRs in 10 HMZ. A measurable difference in contractility of the diseased myocardium is anticipated using invasive hemodynamic measurements that will worsen with age and genotype. These studies will augment the non-invasive imaging studies collected on a larger cohort as discussed above. Collectively, this detailed characterization of the conventional swine model of HF will provide value-add parameters to document the age and severity of cardiac dysfunction required for customers to utilize this tool appropriately within the context of other available models.

### Example 12: Characterize Life-Threatening Arrhythmias According to Disease Severity and RBM20 Genotype

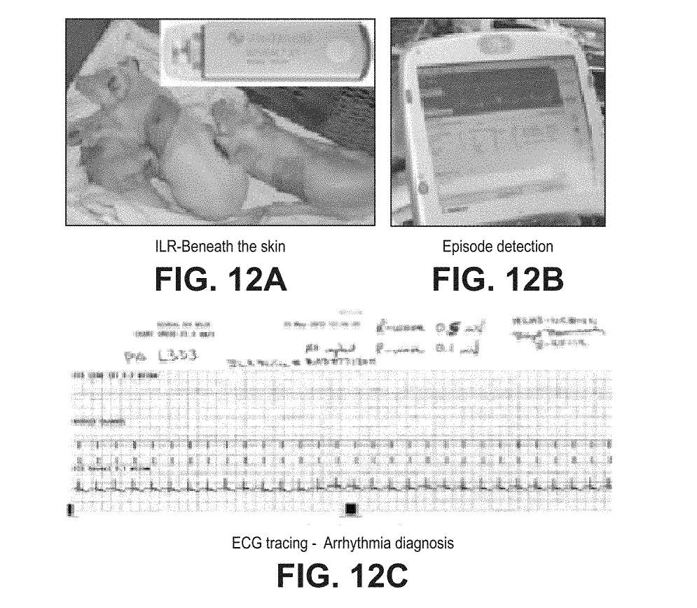

The stress of post-natal life for homozygous RBM20 mutants has proven to provoke sudden death that is presumable due to cardiac dysfunction. The invention now allows investigators to characterize the events leading to death with implantable cardiac monitors including insertable loop recorders (ILRs) such as the Reveal® commercially available from Medtronic, Inc. (FIGS. 12A-12C). This information may provide novel insight into the electrical-mechanical dysfunction in this animal model system of HF for device companies that manufacture pacemakers and internal cardiac defibrillators. As many of these events appear to be early in life, it may be necessary to surgically implant recorders as soon after birth as possible. The surgical procedure implanting ILR devices in the T4 paraspinal region in pigs takes a few minutes and the animals easily tolerate the devices in this location for months. The inventors will begin implanting these devices at the time of birth for all littermates even prior to knowing their individual genotypes. This can be achieved by implementing C-section delivery methods. As propofol is used to sedate the surrogate mother during surgical delivery, the newborn piglets are also fully sedated at the time of birth. Inhaled isoflurane at 1-2% via nose cone is used to keep piglets comfortable and surgically implant the ILRs within minutes after birth. The minor surgical procedure takes no more than 5 minutes per piglet and allows them to recover as they normally would after C-section delivery. This procedure allows all four genotypes (WT, HMZ, HTZ, CMPD) to be collected within each litter. 3-4 sows are used to capture 10-12 animals per genotype. As these devices are not compatible with MRI, this cohort will be used for 8 week hemodynamic studies and will be completed in parallel with Example 7 that will not have implantable devices to ensure proper imaging techniques and analysis.

### Example 13: Measure Hemodynamics in Acute Physiological Stress in Juvenile and Adult RBM20 Genotypes

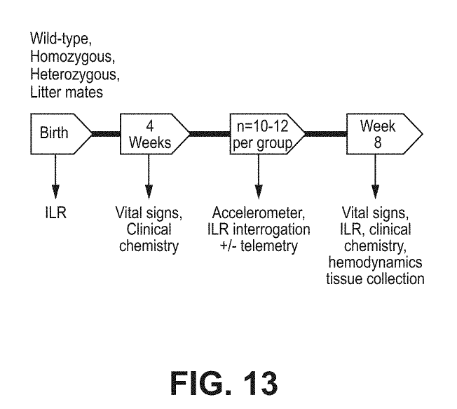

Hemodynamic measurements are the only way to capture myocardial contractile performance directly, which can then be correlated with non-invasive imaging modalities that will be achieved in the characterization of the HF progression in RBM20-R636S. These invasive measurements are important to provide contractility parameters of both the right and left ventricle simultaneously. These invasive hemodynamics will detect subclinical myocardial dysfunction earlier and more accurately than the non-invasive imaging modalities. This is a significant advantage for therapeutics that are focused on mechanical assist devices and/or cardiac resynchronization therapy that benefit from earlier and more quantitative baseline measurements. Hemodynamic measures are documented in both right and left ventricles as well as juvenile and adult animals. This is achieved with terminal experimentation at 8 weeks in the ILR cohort in (FIG. 13) as well as 24 weeks in the MRI imaging cohort in (FIG. 11). This age range is informative to determine the onset of cardiac disease in all four genotypes (including MetS induced WT animals) independent of load and heart rate variables that affect interpretation of non-invasive imaging.

Pressure catheters, such as the Millar Mikro-Tip® (Medtronic, Inc.) paired with data acquisition software such as PowerLabs software (available from ADInstruments, Inc.) are used to generate pressure-volume loops [39]. The right and left side of the heart are be accessed via the pulmonary artery and aorta, using a left thoracotomy, non-survival surgical approach. Cardiac tissue to extract high-quality RNA is also collected at the conclusion of these invasive monitoring studies. Invasive hemodynamic assessment will allow direct measurements of heart rate, mean arterial pressures, end-diastolic volume, end-systolic volume, and calculation of stroke volume and ejection fraction with measured cardiac output. This approach is used to determine the End-diastolic Pressure Volume Relationship (EDPVR) as well as the End-Systolic Pressure Volume Relationship (ESPVR). These PV loops will allow calculations of contractility such as preload recruitable stroke work (PRSW) in response to acute physiological stress [40-42]. The PV loops will be calculated using a standard IVC partial occlusion technique by inflating a balloon catheter accessed through the femoral vein at the conclusion of the acute experimentation. Additionally, atrial pacing is used to induce tachycardia at 120 bpm to measure hemodynamics in the setting of physiological stress. Dobutamine infusion at 2.5-5.0 ug/kg/min may also be used to induce cardiac stress in all three genotypes. This allows for measurements and statistically comparing contractility from direct pressure-volume relationships.

### Example 14: Statistically Analyze and Report Activity of Daily Living (ADL)

Vital signs, ILR, hemodynamics, and histology are statistically analyzed according to previously defined double-blinded protocols [9]. Additionally, preliminary data is obtained using customized accelerometers affixed to ear tags that have documented a significant reduction in spontaneous physical activity for HMZ compared to HTZ litter and pen mates at 10 weeks of age (n=6). Homozygotes averaged 242 min with g-force of 2× or more compared to 551 min in heterozygotes in a 48-hour continuous monitored session. This clinical phenotype of heart failure may provide an additional endpoint to monitor health status of the large animal model system and provide a longitudinal readout of overall exercise capacity. These devices will be used for 48 hour intervals immediately prior to imaging studies. ADL could provide a rapid, cost-effective way to monitor therapeutic efficacy with a hard endpoint of clinical medicine, physical activity.

Engineering HF Mutations into the Ossabaw Miniature Swine Model.

A miniature swine model of genetic HF is used to broaden the scope of applications for device and gene therapy testing. Adult human and adult Ossabaw swine hearts are of equivalent size; a benefit for device testing, and small stature is ideal for cost effective long-term or pharmaceutical studies. Ossabaw swine also develop Metabolic Syndrome (MetS) when fed a high calorie diet, enabling studies with and without this common comorbidity. The equivalency of HF between Ossabaw swine and humans is confirmed upon successful cloning and expansion of this model. This objective enables complimentary study designs not available in conventional swine. This swine model also offer an alternative genetic background if the F2 cloned conventional swine do not stabilize within the HMZ mutant cohort.

### Example 15: Create Ossabaw Fibroblasts with the R636S Genotype by Gene Editing

Male Ossabaw fibroblast HTZ for R636S have been developed (Table 2). Using the methods and reagents demonstrated, female Ossabaw R636S HTZ cells are produced for production of a breeding herd. The cloning efficiency from the selected lines has been high, with a pregnancy rate of 65% and average litter size of 5 liveborn piglets ([33] and unpublished data).

### Example 16: Develop Ossabaw R636S Founders by Cloning

Cells heterozygous for the R636S genotype are cloned. A total of 24 recipient animals are available for the project. Initially 12 rounds of cloning and embryo transfer will be conducted to generate the heterozygous female breeding herd. Considering the typical pregnancy rate of 65% (>70 transfers) with gene-edited cells, a cumulative probability of ˜92% to achieve the goal of 6 or more pregnancies is possible. With a typical litter of 5±2.5, there is a greater than an 80% chance of producing the desired 20 piglets for the genotype. If 5 or fewer pregnancies are established from the first 12 transfers, an additional 6 recipients will be used. To ensure a rejuvenated phenotype of the donor cells for the final six transfers, one pregnancy with the fewest number of fetuses (estimated by ultrasound) is terminated at day 30 to establish rejuvenated fetal fibroblasts [44]. This technique is routinely used in livestock cloning to increase the efficiency of the cloning process. Since few males are required to establish a breeding program, the remaining 6-12 transfers are sufficient to produce healthy breedstock of both heterozygotes and homozygotes. Any remaining embryo transfers could be used to bolster the Ossabaw breeding herd to 30+ animals.

### Example 17: Establish Ossabaw R636S Homozygotes by Breeding

Breeding is conducted as in EXAMPLE 8. At an average of 4 pigs per litter and ˜2 litters per year, an output from the 20-head breeding herd can be expected to break down as follows: HTZ in-cross will produce 80 and 160 HMZ and HTZ per year whereas breeding with a HMZ boar, using the DAZL-surrogate approach, will produce 160 each HMZ and HTZ per year.

### Example 18: Preliminary Histopathology and Molecular Characterization of Ossabaw R636S Mutants

Progression of disease is monitored using at least two methods. 1) Twenty animals with known heterozygous or homozygous genotypes will be monitored monthly by clinical chemistries, ANP and BNP levels. Once a rise in ANP/BNP levels is noted, the subjects (up to 12; n=6 a piece for RBM20 and age-matched Wt respectively) will have cardiac echo, MRI and necropsy performed. 2) Any R636S Ossabaw pig with a failure to thrive phenotype is euthanized for necropsy by veterinary pathologist. Samples of ventricular tissues are excised for molecular characterization and the remaining hearts are fixed for examination by a veterinary pathologist. Plasma levels of ANP and BNP are analyzed to corroborate these biomarkers with histopathology (FIGS. 8A-8F).

### Example 19: Preparation of TITIN Mutants

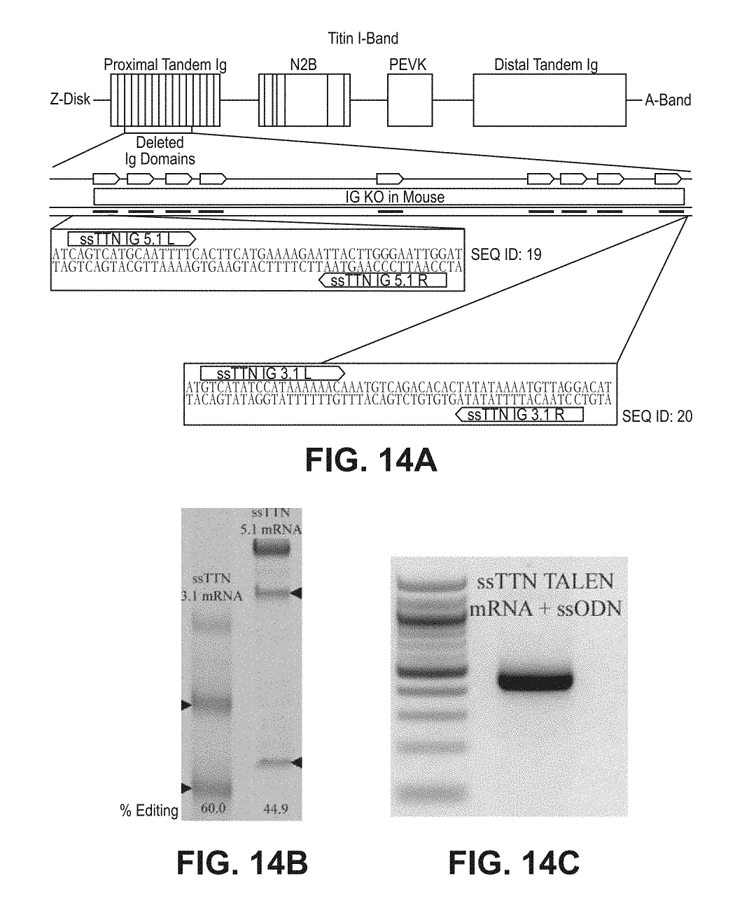

Titin, encoded by the gene TTN, is a protein that is largely responsible for cardiomyocyte passive stiffness. This protein spans from the Z-disk to the M-band of the sarcomere, however, much of the elasticity of the sarcomere is due to the I-band of the titin protein. The I-band is composed of the PEVK and N2B regions, as well as proximal and distal tandem immunoglobulin (Ig) regions. It has been shown that shortening the proximal tandem Ig region leads to a primary diastolic dysfunction phenotype with increased LV stiffness, age-dependent hypertrophy, and exercise intolerance similar to patients with heart failure with preserved ejection fraction¹. FIGS. 14A-14C shows the use of TALENs for making TTN mutants, in this case by excision of the proximal tandem Ig domains. FIG. 14A) TALEN pairs were designed to target the 5′ intron and 3′ intron of Proximal Tandem Ig domains 3 and 11, respectively, of ssTTN. FIG. 14B) Transfected TALEN mRNA targeting either the 5′ intron (5.1) or 3′ intron (3.1) showed an editing efficiency of 44.9% and 60.0% respectively. FIG. 14C) PCR was performed on cells co-transfected with TALEN mRNA and an ssODN repair template designed for the desired allele deletion. The resulting amplicon was the expected size (457 bp) following successful removal of Ig domains 3-11.

Table 5 provides sequences for the TALENs used and the nucleotide sequences they bind to.

TABLE 5

| | | Nucleotide |
|---|---|---|
| TALEN Name | RVD Sequence | Sequence (5′-3′) |
| ssTTN 5.1 L | HD NI NN NG HD | CAGTCATGCAATTTT |
| | NI NG NN HD NI | (SEQ ID: 6) |
| | NI NG NG NG NG | |
| ssTTN 5.1 R | HD HD NI NI NG | CCAATTCCCAAGTAAT |
| | NG HD HD HD NI | (SEQ ID: 7) |
| | NI NN NG NI NI | |
| | NG | |
| ssTTN 3.1 L | NN NG HD NI NG | GTCATATCCATAAAAAAC |
| | NI NG HD HD NI | (SEQ ID: 8) |
| | NG NI NI NI NI | |
| | NI NI HD | |
| ssTTN 3.1 R | NN NG HD HD NG | GTCCTAACATTTTATAT |
| | NI NI HD NI NG | (SEQ ID: 9) |
| | NG NG NG NI NG | |
| | NI NG | |

Table 6 provides a list of primers and sequences used for identification of edited alleles. To test the editing efficiency, the ssTTN Ig 5′ Sense and ssTTN Ig 5′ Antisense or ssTTN Ig 3′ Sense and ssTTN Ig 3′ Antisense were used for the ssTTN 5.1 or ssTTN 3.1 TALEN pair assays, respectively (FIG. 14B). The assay for excision of the Ig region used the ssTTN Ig 5′ Sense and ssTTN IG 3′ Antisense primers (FIG. 14C). As illustrated in FIG. 14B, the two TALENs pairs, 5.1 and 3.1 excise a portion of the proximal tandem Ig domain between from domains 3-11. In order to maintain continuous translation of the entire, modified, Titin protein, the portions of the allele are “stitched” back together using an oligo HDR template comprising: ccttggtatgtgatcagatcagtcatgcaattttcacttcatGGATCCgacacactatataaaatgttaggacatcagctcataaacaga (SEQ ID: 10), where the upper case residues identify a unique restriction site (BamH1, in this case), usable for RFLP analysis along with the primers provided in Table 6, below. The result is a deletion allele with predictable junctions. However, it should be noted that the method does not rely on or require RFLP analysis of the modified allele(s).

TABLE 6

| Primer Name | Primer Sequence (5′-3′) |
|---|---|
| ssTTN Ig 5′ | CTGACCATCGACGCTTCTGA |
| Sense | (SEQ ID: 11) |
| ssTTN IG 5′ | AACTCAACAACGGCACCTGA |
| Antisense | (SEQ ID: 12) |
| ssTTN IG 3′ | CAGATGCGCACCAAAAAGCT |
| Sense | (SEQ ID: 13) |
| ssTTN IG 3′ | CAGCCCTTCCTAATGCCCTC |
| Antisense | (SEQ ID: 14) |

### Example 20: Preparation of BAG3 Mutants

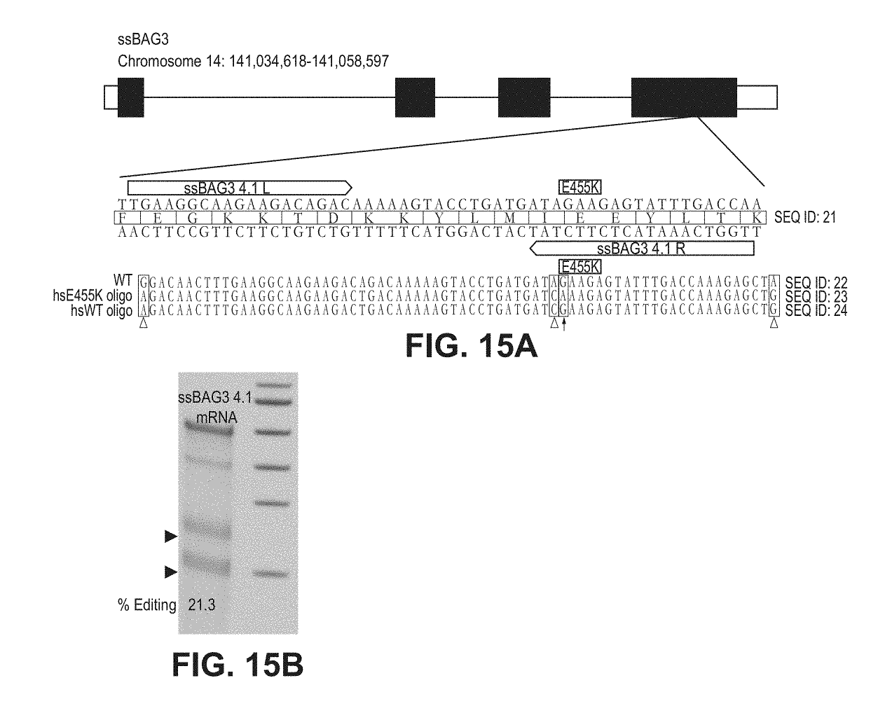

BAG3 is a gene that encodes the Bcl2 associated athanogene 3 protein, which functions as a co-chaperone of heat shock proteins. It has been shown that DCM-associated BAG3 mutations impaired the Z-disc assembly and increased the sensitivities to stress-induced apoptosis¹. The E455K mutation has been shown to reduce BAG domain binding via affinity to all chaperone related proteins via purification-mass spectrometry and more specifically, the mutation maps to a critical region involved in the interaction with heat shock protein Hsp70. FIGS. 15A-15B show the TALENs and E455K humanization template where greyed bases are changed to match the human allele and the E455K mutation is seamlessly introduced. FIG. 15B shows the efficacy of this approach in transfected cells where products denoted by arrowheads are indicative of editing. Table 7 provides a list of TALENs used and the nucleotide sequence they bind.

TABLE 7

| | | Nucleotide |
|---|---|---|
| TALEN Name | RVD Sequence | Sequence (5′-3′) |
| ssBAG3 4.1 L | NG NN NI NI NN | TGAAGGCAAGAAGACAGAC |
| | NN HD NI NI NN | (SEQ ID: 15) |
| | NI NI NN NI HD | |
| | NI NN NI HD | |
| ssBAG3 4.1 R | NG NN NN NG HD | TGGTCAAATACTCTTCTAT |
| | NI NI NI NG NI | (SEQ ID: 16) |
| | HD NG HD NG NG | |
| | HD NG NI NG | |

In terms of HF models, background genetics is known to play a key role in penetrance and severity of disease. As discussed, confounding factors affecting HF include: genetic, congenital heart defects, infections, drug and alcohol abuse, cancer medications, exposure to toxins, coronary artery disease, high blood pressure, diabetes and complications of late-stage pregnancy. The genetic background of the Ossabaw swine is significantly different from that of conventional swine, so it is impossible to predict disease severity in these pigs based on the results in conventional swine. However, since the genes identified in Table 1 have mutations linked to HF in a variety of patient ethnicities, it is expected to be the same in Ossabaw swine with an altered disease course relative to conventional swine. Ossabaw swine are far more inbred than conventional swine, thus less variation is expected in onset and severity of HF in this model. In addition, because MetS can be induced in Ossabaw swine, they provide a unique model with which to identify factors, treatments and interventions in these animals. The reduced size of the Ossabaw pig is advantageous for long-term or interventional testing on adult animals. Therefore, heterozygotes may be preferred. In this case, the breeding herd can consist of mostly wild type females bred to Table 1 modified boars via DAZL surrogates. This will reduce the management and animal welfare burden of maintaining mutant breeding lines.

### Example 21: Comparison of Second Cohort of HMZ Mutants—Analysis of Cardiac Function

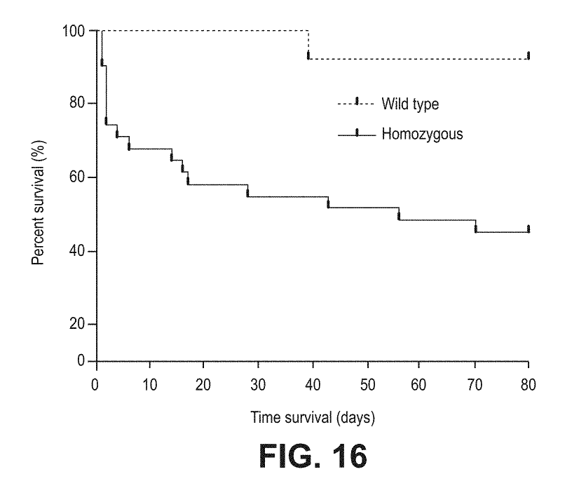

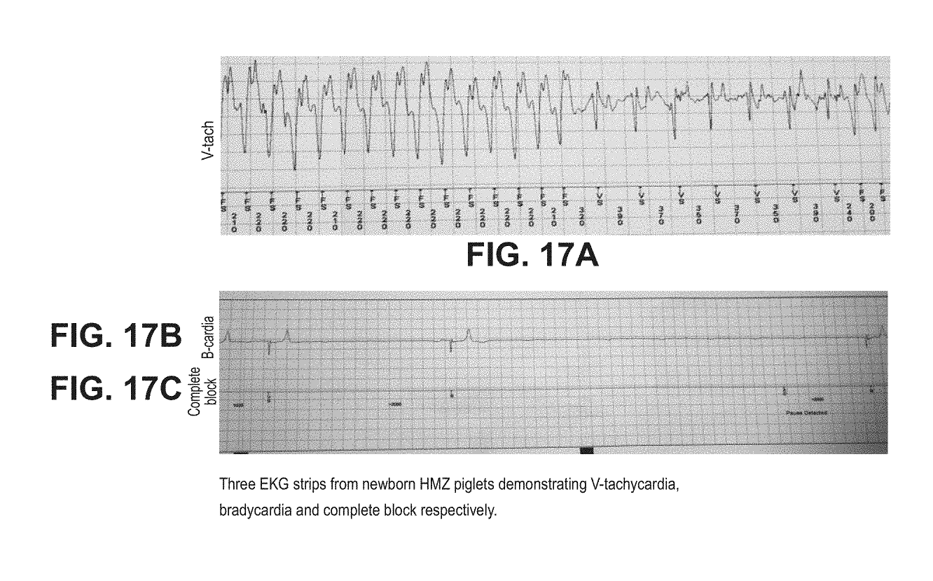

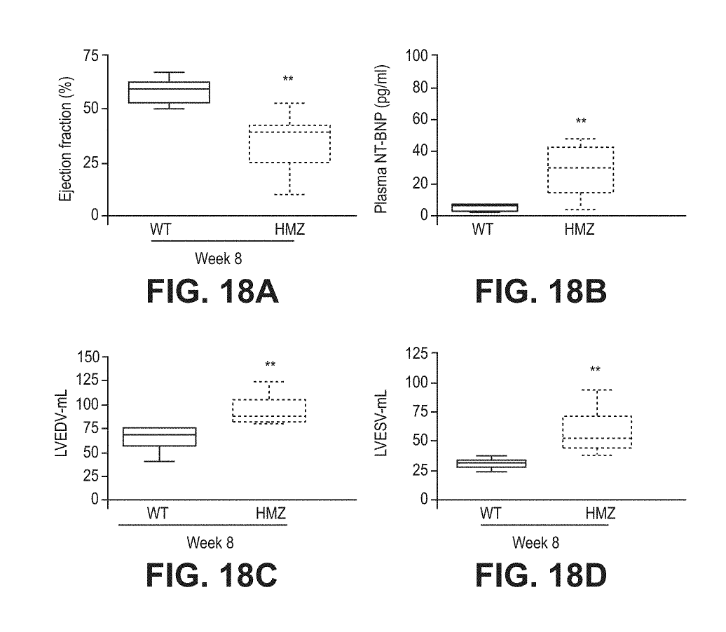

FIGS. 16, 17A-17C and 18A-18D show data from a second cohort of R636S HMZ edited pigs having more intensive animal management practices in light of the high mortality discussed in Example 4 and illustrated in FIG. 4. FIG. 16 provides a further Kaplan Meyer plot of R636S HMZ animals vs. wild-type. Survivability has a tendency to increase with more intensive management practices. FIGS. 17A-17C shows three EKG strips from HMZ piglets showing various symptoms or phenotypes of heart disease. FIG. 17A. ventricular tachycardia; FIG. 17B. bradycardia; and FIG. 17C. complete blockage. FIGS. 18A-18D. Provides a comparison of various measures of cardiac function of WT and R636S HMZ animals. FIG. 18A, shows significant decrease in ejection fraction compared to WT animals; FIG. 18B, shows that R636S animals had significantly increased plasma natriuretic factor compared to WT; FIG. 18C, shows the R636S animals have significantly increased left ventricular end diastolic volume (LVEDV) compared to controls; FIG. 18D illustrates left ventricular end systolic volume (LVESV) is also significantly increased in R636S animals.

Those of skill in the art will appreciate that having HF mutants according to Table 1 in both conventional swine and Ossabaw swine allows for a more complete investigation of CM. For example, conventional swine have much larger litters than Ossabaw swine, e.g., 10-12 vs. 4-5. Also, conventional swine are not pre-disposed to developing metabolic syndrome (MetS). Thus, the ability to provide heterozygotes, compound heterozygotes and homozygotes of mutants may provide a more complete, long-term model for humans with less severe or late onset CM. In contrast, Ossabaw swine are much smaller and therefore more easily handled and have cardiac muscles more similar in size to humans and mature faster making the study of disease pathology easier in these animals. Further, because Ossabaw swine are pre-disposed to developing MetS they provide a further model to the development of this syndrome and methods for its treatment. In addition, because Ossabaw swine are pre-disposed to MetS, heterozygous animals may provide a unique opportunity to study the disease as they syndrome is expected to be less severe and offer a better opportunity for study and treatment of the disease than in homozygous animals which may be too severely affected by the disease for long term studies.

The following paragraphs enumerated consecutively from 1 through 52 provide for various additional aspects of the present invention. In one embodiment, in a first paragraph the invention provides:

1. A genomically modified non-human animal comprising a targeted mutation in one or more genes implicated in heart failure.
2. The genomically modified non-human animal of paragraph 1, wherein the gene is: ANKRD1, BAG3, CAMK2D, CRYAB, CSRP3, DES, DMD, EYA4, GATAD1, ILK, LAMA4, LDB3, LMNA, MYBPC3, MYH6, MYH7, MYPN, PDLIM3, PLN, PSEN1/2, RBM20, RYR2, SCN5A, SGCD, TAZ/G4.5, TCAP, TMPO, TNNC1, TNNI3, TNNT2, TPM1, TTN and/or VCL.
3. The genomically modified animal of any of paragraphs 1 or 2, wherein the mutation is in an RS rich region of a gene.
4. The genomically modified animal of any of paragraphs 1 through 3, wherein the modification is made with gene editing technology.
5. The genomically modified animal of any of paragraphs 1 through 4, wherein the gene editing technology comprises TALENs, CRISPR/CAS9, ZFN, meganucleases.
6. The genomically modified animal of any of paragraphs 1 through 5, wherein the mutation in one or more alleles of one or more genes is the only modification to the genome of the animal.
7. The genomically modified animal of any of paragraphs 1 through 6, wherein the modification is at a specific target locus.
8. The genomically modified animal of any of paragraphs 1 through 7, wherein the animal is a livestock animal.
9. The genomically modified animal of any of paragraphs 1 through 8, wherein the animal is a bovine, ovine or porcine.
10. The genomically modified animal of any of paragraphs 1 through 9, wherein the animal is porcine.
11. The genomically modified animal of any of paragraphs 1 through 10, wherein the porcine animal is a minipig.
12. The genomically modified animal of any of paragraphs 1 through 11, wherein the minipig is an Ossabaw minipig
13. The genomically modified animal of any of paragraphs 1 through 12, wherein the modification is heterozygous.
14. The genomically modified animal of any of paragraphs 1 through 13, wherein the modification is homozygous.
15. The genomically modified animal of any of paragraphs 1 through 14, wherein the modification is compound homozygous.
16. The genomically modified animal of any of paragraphs 1 through 15, wherein the modification in the RBM allele comprises R636H, R636S or S635A; wherein the modification of the BAGS allele comprises E455K or wherein the modification in the TTN allele comprises a deletion of an Ig domain.
17. The genomically modified animal of any of paragraphs 1 through 16, wherein the animal develops right and left heart dysfunction together.
18. The genomically modified animal of any of paragraphs 1 through 17, wherein the animal develops right and left dysfunction separately.
19. The genomically modified animal of any of paragraphs 1 through 18, wherein multiple gene are modified in serial.
20. The genomically modified animal of any of paragraphs 1 through 19, wherein multiple genes are modified in tandem using multiplex gene editing.
21. A method of making a non-human, animal-model for heart failure, comprising modifying an animal genome to target modifications in one or more genes indicated in cardiomyopathy.
22. The method of paragraph 21, wherein the gene is: ANKRD1, BAGS, CAMK2D, CRYAB, CSRP3, DES, DMD, EYA4, GATAD1, ILK, LAMA4, LDB3, LMNA, MYBPC3, MYH6, MYH7, MYPN, PDLIM3, PLN, PSEN1/2, RBM20, RYR2, SCN5A, SGCD, TAZ/G4.5, TCAP, TMPO, TNNC1, TNNI3, TNNT2, TPM1, TTN and/or VCL.
23. The method of any of paragraphs 21 or 22, wherein the modification is site-specific.
24. The method of any of paragraphs 21 through 23, wherein only the genes targeted are modified.
25. The method of any of paragraphs 21 through 24, wherein the mutation is within a hotspot in the gene.
26. The method of any of paragraphs 21 through 25, wherein the modification is in the RS rich region of a gene.
27. The method of any of paragraphs 21 through 26, wherein the modification is made with gene editing technology.
28. The method of any of paragraphs 21 through 27, wherein the gene editing technology comprises TALENs, CRISPR/CAS9. ZFN, meganucleases.
29. The method of any of paragraphs 21 through 28, wherein the modification in the allele is the only modification to the genome of the animal.
30. The method of any of paragraphs 21 through 29, wherein the animal is a livestock animal.
31. The method of any of paragraphs 21 through 30, wherein the animal is a goat, bovine, ovine or porcine.
32. The method of any of paragraphs 21 through 31, wherein the animal is porcine.
33. The method of any of paragraphs 21 through 32, wherein the porcine animal is a minipig.
34. The method of any of paragraphs 21 through 33, wherein the minipig is an Ossabaw minipig.
35. The method of any of paragraphs 21 through 34, wherein the modification is heterozygous.
36. The method of any of paragraphs 21 through 35, wherein the modification is homozygous.
37. The method of any of paragraphs 21 through 36, wherein the modification is compound heterozygous.
38. The method of any of paragraphs 21 through 37, wherein the modification is R636H, R636S or S635A of RBM20.
39. The method of any of paragraphs 21 through 38, wherein the animal develops right and left heart dysfunction together.
40. The method of any of paragraphs 21 through 39, wherein the animal develops right and left heart dysfunction separately.
41. The method of any of paragraphs 21 through 40, wherein the method provides a suite of animals comprising heterozygous, compound heterozygotes and homozygotes for a modification.
42. An animal model for heart disease comprising a non-human animal comprising a targeted modification of one or more genes indicated in heart disease.
43. The animal model of paragraph 42, wherein the gene comprises ANKRD1, BAG3, CAMK2D, CRYAB, CSRP3, DES, DMD, EYA4, GATAD1, ILK, LAMA4, LDB3, LMNA, MYBPC3, MYH6, MYH7, MYPN, PDLIM3, PLN, PSEN1/2, RBM20, RYR2, SCN5A, SGCD, TAZ/G4.5, TCAP, TMPO, TNNC1, TNNI3, TNNT2, TPM1, TIN and/or VCL
44. The animal model of any of paragraphs 42 or 43, where in the genetic modification is accomplished by gene editing technology.
45. The animal model of any of paragraphs 42 through 44, wherein the genetic modification is the only modification to the animal.
46. The animal model of any of paragraphs 42 through 45, wherein the gene editing technology includes TALENs, zinc finger nucleases (ZFN), meganuclease or CRISPR/CAS.
47. The animal model of any of paragraphs 42 through 46, wherein the modification is site-specific.
48. The animal model of any of paragraphs 42 through 47, wherein the animal is used in clinical testing of drugs, biologics or devices to treat heart failure.
49. The animal model of paragraph 42 through 48, wherein the model comprises WT, homozygotes, heterozygotes and compound heterozygotes.
50. A genetically modified pig as a model for studying heart disease wherein the genome of the modified prig comprises at least one modified gene or combination of modified genes selected from:

i) human ANKRD1, BAG3, CAMK2D, CRYAB, CSRP3, DES, DMD, EYA4, GATAD1, ILK, LAMA4, LDB3, LMNA, MYBPC3, MYH6, MYH7, MYPN, PDLIM3, PLN, PSEN1/2, RBM20, RYR2, SCN5A, SGCD, TAZ/G4.5, TCAP, TMPO, TNNC1, TNNI3, TNNT2, TPM1, TTN and/or VCL.; and/or

ii) pig ANKRD1, BAG3, CAMK2D, CRYAB, CSRP3, DES, DMD, EYA4, GATAD1, ILK, LAMA4, LDB3, LMNA, MYBPC3, MYH6, MYH7, MYPN, PDLIM3, PLN, PSEN1/2, RBM20, RYR2, SCN5A, SGCD, TAZ/G4.5, TCAP, TMPO, TNNC1, TNNI3, TNNT2, TPM1, TTN and/or VCL wherein the modified pig expresses at least one phenotype associated with heart disease.

51. The genetically modified pig of any of the preceding paragraphs, wherein the symptom is ventricular tachycardia, dilated cardiomyopathy (DCM), hypertrophic cardiomyopathy (HCM), restrictive cardiomyopathy (RCM), arrhythmogenic cardiomyopathy (AVC) and unclassified cardiomyopathy.
52. The genetically modified pig of any of the preceding paragraphs, wherein the phenotype is ventricular tachycardia, ventricular bradycardia, arrhythmia, ventricular blockage, and/or abnormal cardiac function LVEF, LVEDV, SV, LVESV and NT-BNP.
53. The genetically modified pig of any of the preceding paragraphs, wherein the genetic modification is accomplished by gene editing.

All patents, publications, and journal articles set forth herein are hereby incorporated by reference herein; in case of conflict, the instant specification is controlling.

While this invention has been described in conjunction with the various exemplary embodiments outlined above, various alternatives, modifications, variations, improvements, and/or substantial equivalents, whether known or that are or may be presently unforeseen, may become apparent to those having at least ordinary skill in the art. Accordingly, the exemplary embodiments according to this invention, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents of these exemplary embodiments.

1. A genomically modified non-human animal comprising a targeted mutation in one or more genes implicated in heart failure.

2. The genomically modified non-human animal of claim 1, wherein the gene is: ANKRD1, BAG3, CAMK2D, CRYAB, CSRP3, DES, DMD, EYA4, GATAD1, ILK, LAMA4, LDB3, LMNA, MYBPC3, MYH6, MYH7, MYPN, PDLIM3, PLN, PSEN1/2, RBM20, RYR2, SCN5A, SGCD, TAZ/G4.5, TCAP, TMPO, TNNC1, TNNI3, TNNT2, TPM1, TTN and/or VCL

3. The genomically modified animal of claim 1, wherein the mutation is in an RS rich region of a gene.

4. The genomically modified animal of claim 1, wherein the modification is made with gene editing technology.

5. The genomically modified animal of claim 4, wherein the gene editing technology comprises TALENs, CRISPR/CAS9, ZFN, meganucleases.

6. The genomically modified animal of claim 1, wherein the mutation in one or more alleles of one or more genes is the only modification to the genome of the animal.

7. The genomically modified animal of claim 6, wherein the modification is at a specific target locus.

8. The genomically modified animal of claim 1, wherein the animal is a livestock animal.

9. The genomically modified animal of claim 8, wherein the animal is a bovine, ovine or porcine.

10. The genomically modified animal of claim 9, wherein the animal is porcine.

11. The genomically modified animal of claim 10, wherein the porcine animal is a minipig.

12. The genomically modified animal of claim 11, wherein the minipig is an Ossabaw minipig

13. The genomically modified animal of claim 1, wherein the modification is heterozygous.

14. The genomically modified animal of claim 1, wherein the modification is homozygous.

15. The genomically modified animal of any of claim 1, wherein the modification is compound homozygous.

16. The genomically modified animal of any of claim 1, wherein the modification in the RBM allele comprises R636H, R636S or S635A; wherein the modification of the BAGS allele comprises E455K or wherein the modification in the TTN allele comprises a deletion of an Ig domain.

17. The genomically modified animal of any of claim 1, wherein the animal develops right and left heart dysfunction together.

18. The genomically modified animal of any of claim 1, wherein the animal develops right and left dysfunction separately.

19. The genomically modified animal of any of claim 1, wherein multiple gene are modified in serial.

20. The genomically modified animal of any of claim 1, wherein multiple genes are modified in tandem using multiplex gene editing.

21. A method of making a non-human, animal-model for heart failure, comprising modifying an animal genome to target modifications in one or more genes indicated in cardiomyopathy.

22. The method of claim 21, wherein the gene is: ANKRD1, BAGS, CAMK2D, CRYAB, CSRP3, DES, DMD, EYA4, GATAD1, ILK, LAMA4, LDB3, LMNA, MYBPC3, MYH6, MYH7, MYPN, PDLIM3, PLN, PSEN1/2, RBM20, RYR2, SCN5A, SGCD, TAZ/G4.5, TCAP, TMPO, TNNC1, TNNI3, TNNT2, TPM1, TIN and/or VCL

23. The method of any of claim 21, wherein the modification is site-specific.

24. The method of any of claim 21, wherein only the genes targeted are modified.

25. The method of any of claim 21, wherein the mutation is within a hotspot in the gene.

26. The method of any of claim 21, wherein the modification is in the RS rich region of a gene.

27. The method of any of claim 21, wherein the modification is made with gene editing technology.

28. The method of any of claim 27, wherein the gene editing technology comprises TALENs, CRISPR/CAS9. ZFN, meganucleases.

29. The method of any of claim 21, wherein the modification in the allele is the only modification to the genome of the animal.

30. The method of any of claim 21, wherein the animal is a livestock animal.

31. The method of any of claim 21, wherein the animal is a bovine, ovine or porcine.

32. The method of any of claim 21, wherein the animal is porcine.

33. The method of any of claim 32, wherein the porcine animal is a minipig.

34. The method of any of claim 33, wherein the minipig is an Ossabaw minipig

35. The method of any of claim 21, wherein the modification is heterozygous.

36. The method of any of claim 21, wherein the modification is homozygous.

37. The method of any of claim 21, wherein the modification is compound heterozygous.

38. The method of any of claim 21, wherein the modification is R636H, R636S or S635A of RBM20.

39. The method of any of claim 21, wherein the animal develops right and left heart dysfunction together.

40. The method of any of claim 21, wherein the animal develops right and left heart dysfunction separately.

41. The method of any of claim 21, wherein the method provides a suite of animals comprising heterozygous, compound heterozygotes and homozygotes for a modification.

42. An animal model for heart disease comprising a non-human animal comprising a targeted modification of one or more genes indicated in heart disease.

43. The animal model of claim 42, wherein the gene comprises ANKRD1, BAG3, CAMK2D, CRYAB, CSRP3, DES, DMD, EYA4, GATAD1, ILK, LAMA4, LDB3, LMNA, MYBPC3, MYH6, MYH7, MYPN, PDLIM3, PLN, PSEN1/2, RBM20, RYR2, SCN5A, SGCD, TAZ/G4.5, TCAP, TMPO, TNNC1, TNNI3, TNNT2, TPM1, TTN and/or VCL.

44. The animal model of any of claim 42, where in the genetic modification is accomplished by gene editing technology.

45. The animal model of any of claim 42, wherein the genetic modification is the only modification to the animal.

46. The animal model of any of claim 44, wherein the gene editing technology includes TALENs, zinc finger nucleases (ZFN), meganuclease or CRISPR/CAS.

47. The animal model of any of claim 42, wherein the modification is site-specific.

48. The animal model of any of claim 42, wherein the animal is used in clinical testing of drugs, biologics or devices to treat heart failure.

49. The animal model of claim 42, wherein the model comprises WT, homozygotes, heterozygotes and compound heterozygotes.

50. A genomically modified pig as a model for studying heart disease wherein the genome of the modified prig comprises at least one modified gene or combination of modified genes selected from:
i) human ANKRD1, BAG3, CAMK2D, CRYAB, CSRP3, DES, DMD, EYA4, GATAD1, ILK, LAMA4, LDB3, LMNA, MYBPC3, MYH6, MYH7, MYPN, PDLIM3, PLN, PSEN1/2, RBM20, RYR2, SCN5A, SGCD, TAZ/G4.5, TCAP, TMPO, TNNC1, TNNI3, TNNT2, TPM1, TTN and/or VCL; and/or
ii) pig ANKRD1, BAG3, CAMK2D, CRYAB, CSRP3, DES, DMD, EYA4, GATAD1, ILK, LAMA4, LDB3, LMNA, MYBPC3, MYH6, MYH7, MYPN, PDLIM3, PLN, PSEN1/2, RBM20, RYR2, SCN5A, SGCD, TAZ/G4.5, TCAP, TMPO, TNNC1, TNNI3, TNNT2, TPM1, TTN and/or VCL wherein the modified pig expresses at least one phenotype associated with heart disease.

51. The genomically modified pig of claim 50, wherein the symptom is ventricular tachycardia, dilated cardiomyopathy (DCM), hypertrophic cardiomyopathy (HCM), restrictive cardiomyopathy (RCM), arrhythmogenic cardiomyopathy (AVC) and unclassified cardiomyopathy.

52. The genomically modified pig of claim 50, wherein the phenotype is ventricular tachycardia, bradycardia, arrhythmia, cardiac blockage, and/or abnormal cardiac function.

53. The genomically modified pig of claim 50, wherein the genetic modification is accomplished by gene editing.
